(19) United States
(12) Patent Application Publication
(10) Pub. No.: US 2020/0281708 A1
(43) Pub. Date: Sep. 10, 2020

(54) SYSTEMS AND METHODS FOR PROTECTING THE CEREBRAL VASCULATURE

(71) Applicant: BOSTON SCIENTIFIC SCIMED, INC., Maple Grove, MN (US)

(72) Inventors: Antony J. Fields, San Francisco, CA (US); Shih-hsiung Albert Yuan, Pleasanton, CA (US); Whittaker Ian Hamill, Petaluma, CA (US); Daniel W. Fifer, Windsor, CA (US)

(73) Assignee: BOSTON SCIENTIFIC SCIMED, INC., MAPLE GROVE, MN (US)

(21) Appl. No.: 16/807,658

(22) Filed: Mar. 3, 2020

Related U.S. Application Data
(60) Provisional application No. 62/813,684, filed on Mar. 4, 2019.

(51) Int. Cl. A61F 2/01; A61B 34/20

(52) U.S. Cl. CPC A61F 2/01; A61B 34/20; A61F 2002/011; A61M 25/09; A61F 2002/016; A61F 2250/0098; A61F 2210/0014

(57) ABSTRACT

Vascular filters and deflectors and methods for filtering bodily fluids. A blood filtering assembly can capture embolic material dislodged or generated during an endovascular procedure to inhibit or prevent the material from entering the cerebral vasculature. A single vascular filter may be positionable within the aorta to protect all four cerebral arteries.

### RELATED APPLICATIONS

This application claims the benefit of priority from U.S. Provisional Application No. 62/813,684 filed Mar. 4, 2019, which is incorporated herein by reference.

### TECHNICAL FIELD

In general, the present disclosure relates to medical devices for filtering blood. And, more particularly, in certain embodiments, to a method and a system of filters and deflectors for protecting the cerebral arteries from emboli, debris and the like dislodged during an endovascular or cardiac procedure.

### BACKGROUND

There are four arteries that carry oxygenated blood to the brain, i.e., the right and left vertebral arteries, and the right and left common carotid arteries. Various procedures conducted on the human body, e.g., transcatheter aortic valve replacement (TAVR), aortic valve valvuloplasty, carotid artery stenting, closure of the left atrial appendage, mitral valve annuloplasty, repair or replacement, can cause and/or dislodge materials (whether native or foreign), these dislodged bodies can travel into one or more of the cerebral arteries resulting in, inter alia, stroke. Moreover, atheromas along and within the aorta and aortic arch can be dislodged as the TAVR catheter is advanced toward the diseased aortic valve and subsequently withdrawn after implantation is completed. In addition, pieces of the catheter itself can be stripped away during delivery and implantation. These various forms of vascular debris, whether native or foreign, can then travel into one or more cerebral arteries, embolize and cause, inter alia, a stroke or strokes.

There exist devices for protecting one or more cerebral arteries by either collecting (filters) or deflecting (deflectors) debris. Of the known medical devices, delivery systems, and methods, each has certain advantages and disadvantages. There is an ongoing need to provide alternative medical devices and methods as well as alternative methods for manufacturing and using medical devices.

### SUMMARY

Vascular filters and deflectors and methods for filtering bodily fluids are disclosed herein. A blood filtering assembly can capture embolic material dislodged or generated during an endovascular procedure to inhibit or prevent the material from entering the cerebral vasculature. A blood deflecting assembly can deflect embolic material dislodged or generated during an endovascular procedure to inhibit or prevent the material from entering the cerebral vasculature.

In a first example, an embolic protection system for isolating cerebral vasculature may comprise a protection device having a proximal portion configured to remain outside the body and a distal portion. The distal portion may comprise an outer sheath and an expandable filter assembly comprising a variable diameter frame and a filter element.

Alternatively or additionally to any of the examples above, in another example, the variable diameter frame may comprise an inflatable tube.

Alternatively or additionally to any of the examples above, in another example, the inflatable tube may comprise an inflation cavity and a valve.

Alternatively or additionally to any of the examples above, in another example, the inflatable tube may be fluidly coupled to an inflation lumen configured to extend proximally from the expandable filter assembly to the proximal portion of the protection device.

Alternatively or additionally to any of the examples above, in another example, the variable diameter frame may comprise an open spaced coil including a plurality of windings.

Alternatively or additionally to any of the examples above, in another example, the variable diameter frame may comprise a laser cut tube having a plurality of openings.

Alternatively or additionally to any of the examples above, in another example, the variable diameter frame may comprise a shape memory material.

Alternatively or additionally to any of the examples above, in another example, the embolic protection system may further comprise a tension element extending through a central lumen of the variable diameter frame.

Alternatively or additionally to any of the examples above, in another example, a distal end of the tension element may be fixedly coupled to the variable diameter frame and a proximal end may extend proximally from the variable diameter frame.

Alternatively or additionally to any of the examples above, in another example, a proximal force exerted on the proximal end of the tension element may be configured to reduce a diameter of the variable diameter frame.

Alternatively or additionally to any of the examples above, in another example, the expandable filter assembly may comprise a distally facing opening.

Alternatively or additionally to any of the examples above, in another example, the expandable filter assembly may further comprise a support element.

Alternatively or additionally to any of the examples above, in another example, the support element may be one or more longitudinally extending tines.

Alternatively or additionally to any of the examples above, in another example, the support element may be an elongated hoop positioned between the variable diameter frame and a base of the filter element.

Alternatively or additionally to any of the examples above, in another example, the expandable filter assembly may further comprise a tether coupled to an end of the filter element.

In another example, a method of inhibiting embolic material from entering cerebral vasculature may comprise positioning a guidewire through a right subclavian artery and into an aortic arch and tracking a distal portion of a protection device over the guidewire. The distal portion of the protection device may comprise a proximal sheath, a proximal self-expanding filter assembly radially within the proximal sheath, a distal sheath, and a distal self-expanding filter assembly radially within the distal sheath. At least one of proximally retracting the proximal sheath and distally advancing the proximal self-expanding filter assembly may deploy the proximal self-expanding filter assembly from the proximal sheath in an innominate artery. The method may further comprise steering the distal sheath into the aortic arch and at least one of proximally retracting the distal sheath and distally advancing the distal self-expanding filter assembly to deploy the distal self-expanding filter assembly from the distal sheath in the aortic arch. After deploying the proximal self-expanding filter assembly and distal self-expanding filter assembly, the method may further comprise withdrawing the proximal sheath and the distal sheath.

Alternatively or additionally to any of the examples above, in another example, an opening of the distal self-expanding filter assembly may be positioned in the aortic arch upstream of an ostium of a left common carotid artery.

Alternatively or additionally to any of the examples above, in another example, the opening may be a distally facing opening.

Alternatively or additionally to any of the examples above, in another example, the distal self-expanding filter assembly may comprise a frame, a filter element, and a support element.

Alternatively or additionally to any of the examples above, in another example, the support element may be one or more longitudinally extending tines.

Alternatively or additionally to any of the examples above, in another example, the support element may be an elongated hoop positioned between the frame and a base of the filter element.

In another example, a method of inhibiting embolic material from entering cerebral vasculature may comprise positioning a guidewire through a right subclavian artery and into an ascending aorta and tracking a distal portion of a protection device over the guidewire. The distal portion of the protection device may comprise an outer sheath and a self-expanding filter assembly radially within the outer sheath. At least one of proximally retracting the outer sheath and distally advancing the self-expanding filter assembly may deploy the self-expanding filter assembly from the outer sheath in the ascending aorta.

Alternatively or additionally to any of the examples above, in another example, the self-expanding filter assembly may comprise a frame, a filter element, and a support element.

Alternatively or additionally to any of the examples above, in another example, the support element may be one or more longitudinally extending tines.

Alternatively or additionally to any of the examples above, in another example, the support element may be an elongated hoop positioned between the frame and a base of the filter element.

Alternatively or additionally to any of the examples above, in another example, the self-expanding filter assembly further may comprise a tether coupled to a base of a filter element.

Alternatively or additionally to any of the examples above, in another example, proximal actuation of the tether may draw the base of the filter element into the outer sheath.

Alternatively or additionally to any of the examples above, in another example, the self-expanding filter assembly may comprise an elongated tubular body.

Alternatively or additionally to any of the examples above, in another example, the elongated tubular body may comprise one or more woven, braided, or knitted filaments.

Alternatively or additionally to any of the examples above, in another example, a distal end of the elongated tubular body may comprise a hem.

In another example, a method of inhibiting embolic material from entering cerebral vasculature may comprise positioning a guidewire through a right subclavian artery and into an ascending aorta and tracking a distal portion of a protection device over the guidewire. The distal portion of the protection device may comprise an outer sheath and an inflatable filter assembly radially within the outer sheath. The method may further comprise at least one of proximally retracting the outer sheath and distally advancing the inflatable filter assembly to deploy the inflatable filter assembly from the outer sheath in the ascending aorta and delivering an inflation fluid to the inflatable filter assembly to expand a frame of the inflatable filter assembly.

Alternatively or additionally to any of the examples above, in another example, the frame of the inflatable filter assembly may comprise an inflation cavity and a valve.

Alternatively or additionally to any of the examples above, in another example, the inflation cavity may be fluidly coupled to an inflation lumen.

Alternatively or additionally to any of the examples above, in another example, the inflatable filter assembly may further comprise a filter element coupled to the frame.

Alternatively or additionally to any of the examples above, in another example, the method may further comprise removing the inflation fluid.

The above summary of exemplary embodiments is not intended to describe each disclosed embodiment or every implementation of the present disclosure.

### BRIEF DESCRIPTION OF THE DRAWINGS

The invention may be more completely understood in consideration of the following detailed description of various embodiments in connection with the accompanying drawings, in which:

FIG. 1 illustrates a filter assembly to protect the cerebral vascular architecture;

FIG. 2 illustrates an alternate embodiment of a filter assembly to protect the cerebral vascular architecture;

FIG. 3 illustrates another alternate embodiment a filter assembly to protect the cerebral vascular architecture;

FIG. 4 illustrates another alternate embodiment a filter assembly to protect the cerebral vascular architecture;

FIG. 5 illustrates another alternate embodiment a filter assembly to protect the cerebral vascular architecture;

FIG. 6 illustrates another alternate embodiment a filter assembly to protect the cerebral vascular architecture;

FIG. 7 illustrates another alternate embodiment a filter assembly to protect the cerebral vascular architecture; and

FIG. 8 illustrates another alternate embodiment a filter assembly to protect the cerebral vascular architecture.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

The currently marketed Sentinel system made by Claret Medical and embodiments of which are described in U.S. Pat. No. 9,492,264 mentioned above has two filters, a first which protects the right brachiocephalic artery, from which the right vertebral and right common carotid arteries typically originate, and a second filter in the left common carotid artery. In a typical patient, the left vertebral artery which provides approximately seven percent of the perfusion to the brain is left unprotected.

One disclosed solution to protecting the left vertebral is the use of a second device intended to be placed in the left arm, e.g. through the left radial artery, with a filter placed in the left subclavian from which the left vertebral typically originates. Embodiments of such a solution can be found in U.S. Pat. No. 9,566,144, the entirety of which is hereby incorporated by reference herein.

While cerebral embolic protection for transcatheter aortic valve replacement (TAVR) procedures may require that the embolic protection device allow for the passage of procedural catheters and devices over the aortic arch, procedures where the procedural catheters are introduced through the right heart or directly into the apex of the heart may allow for an embolic protection device to be positioned within the aorta or aortic arch. For example, procedures such as, but not limited to, left atrial appendage occlusion (LAAO), transcatheter mitral valve repair (TMVR), transcatheter mitral valve replacement, TAVR via apical access, ablation for afibrillation, ablation for ventricular tachycardia, etc. may allow for one or more embolic protection device(s) to be positioned upstream of the four cerebral arteries. The present application discloses several embodiments which may include a single filter and/or compound systems of filters and/or deflectors that can provide full cerebral protection.

The disclosure generally relates to devices and methods for filtering fluids and/or deflecting debris contained within fluids, including body fluids such as blood. A filtering or deflecting device can be positioned in an artery before and/or during an endovascular procedure (e.g., transcatheter aortic valve implantation (TAVI) or replacement (TAVR), transcatheter mitral valve implantation (TAMI) or replacement (TAMR), surgical aortic valve replacement (SAVR), other surgical valve repair, implantation, or replacement, cardiac ablation (e.g., ablation of the pulmonary vein to treat atrial fibrillation) using a variety of energy modalities (e.g., radio frequency (RF), energy, cryo, microwave, ultrasound), cardiac bypass surgery (e.g., open-heart, percutaneous), transthoracic graft placement around the aortic arch, valvuloplasty, etc.) to inhibit or prevent embolic material such as debris, emboli, thrombi, etc. resulting from entering the cerebral vasculature.

The devices may be used to trap and/or deflect particles in other blood vessels within a subject, and they can also be used outside of the vasculature. The devices described herein are generally adapted to be delivered percutaneously to a target location within a subject, but can be delivered in any suitable way and need not be limited to minimally-invasive procedures.

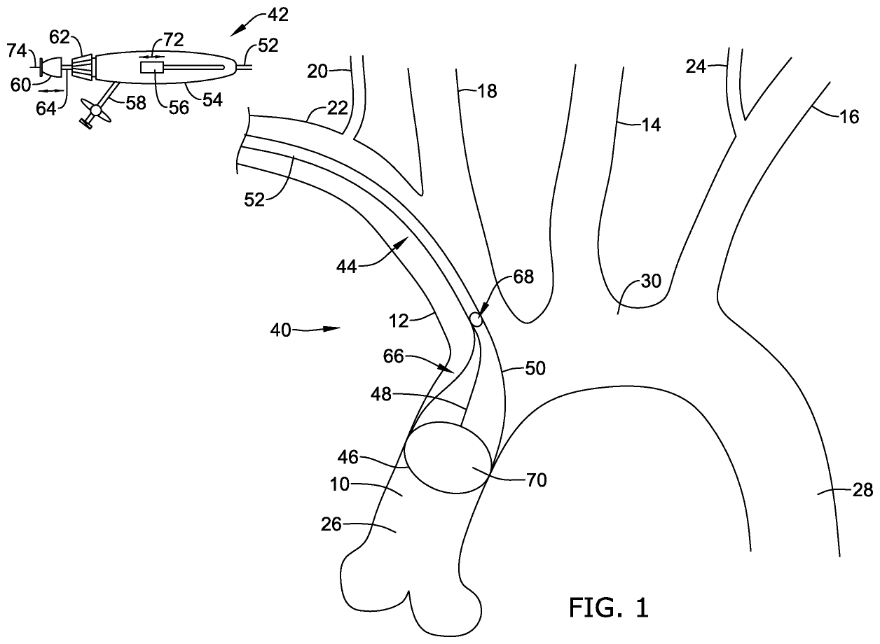

FIG. 1 is a schematic view of a portion of an aorta 10 including a protection system 40. The aorta includes the ascending aorta 26, descending aorta 28, and aortic arch 30. The aortic arch 30 is upstream of the left and right coronary arteries (not explicitly shown). The aorta 10 typically includes three great branch arteries: the brachiocephalic artery or innominate artery 12, the left common carotid artery 14, and the left subclavian artery 16. The innominate artery 12 branches to the right carotid artery 18, then the right vertebral artery 20, and thereafter is the right subclavian artery 22. The right subclavian artery 22 supplies blood to, and may be directly accessed from (termed right radial access), the right arm. The left subclavian artery 16 branches to the left vertebral artery 24, usually in the shoulder area. The left subclavian artery 16 supplies blood to, and may be directly accessed from (termed left radial axis), the left arm. Four of the arteries illustrated in FIG. 1 supply blood to the cerebral vasculature: (1) the left common carotid artery 14 (about 40% of cerebral blood supply); (2) the right common carotid artery 18 (about 40% of cerebral blood supply); (3) the right vertebral artery 20 (about 10% of cerebral blood supply); and (4) the left vertebral artery 24 (about 10% of cerebral blood supply).

It may be desirable to filter blood flow to all four arteries 14, 18, 20, 24 supplying blood to the brain and/or deflect particulates from entering the arteries 14, 18, 20, 24 supplying the brain. It may also be desirable to limit the number of incision sites or cuts required to deploy the system(s). FIG. 1 illustrates deploying the protection system 40 using a right radial access incision. However, it is contemplated that the protection system 40 may deployed using a left radial access incision, a femoral incision, or other location, as desired.

The protection system 40 may include a proximal portion 42 and a distal portion 44. The proximal portion 42 is configured to be held outside a patient's body and manipulated by a user such as a surgeon. The distal portion 44 is configured to be positioned such that a filter assembly 66 is located at a target location such as the ascending aorta 26 (or other location upstream of the four cerebral arteries 14, 18, 20, 24) to remove debris prior to reaching the brain and other distal organs. In some embodiments, the filter assembly 66 may be placed in the ascending aorta 26 between the aortic root (not explicitly shown) and the ostium of the innominate artery 12.

The proximal portion 42 comprises a handle 54, a control 56 such as a slider, an outer sheath 52, a port 58, optionally, an inner member translation control 60 such as a knob, and optionally, a hemostasis valve control 62 such as a knob. The proximal portion 42 may also comprise an inner member 64 radially inward of the outer sheath 52. The proximal portion 42 may also comprise a filter wire 48 radially inward of the outer sheath 52. The filter wire 48 is coupled to the filter assembly 66 at the distal portion 44. The outer sheath 52 may have a diameter between about 4 French (Fr) (approximately 1.33 millimeters (mm)) and about 6 Fr (approximately 2 mm). The outer sheath 52 may have a diameter smaller than 4 Fr or greater than 6 Fr depending on the application. The outer sheath 52 may comprise an atraumatic distal tip. Other features of the protection system 40 and other protection systems described herein may be flexible and/or atraumatic. The outer sheath 52 may comprise a curvature, for example based on an intended placement location (e.g., the ascending aorta 26).

The slider 56 can be used to translate the outer sheath 52 and/or a filter assembly 66 (e.g., coupled to the filter wire 48). For example, the slider 56 may proximally retract the outer sheath 52, the slider 56 may distally advance the filter assembly 66 out of the outer sheath 52, or the slider 56 may proximally retract the outer sheath 52 and distally advance the filter assembly 66 (e.g., simultaneously or serially), which can allow the filter assembly 66 to radially expand. The slider 56 may also be configured to have an opposite translation effect, which can allow the filter assembly 66 to be radially collapsed (e.g., due to compression by the outer sheath 52) as the filter assembly 66 is drawn into the outer sheath 52. Other deployment systems are also possible. For example, other deployment systems for opening and/or closing the filter assembly 66 may include, but are not limited to, gears or other features such as helical tracks (e.g., configured to compensate for any differential lengthening due to foreshortening of the filter assembly 66 and/or configured to convert rotational motion into longitudinal motion), a mechanical element, a pneumatic element, a hydraulic element, etc.

The port 58 may be in fluid communication with the inner member 64 (e.g., via a Y-shaped connector in the handle 54). The port 58 can be used to flush the device (e.g., with saline) before, during, and/or after use, for example, to remove air. The port 58 can also or alternatively be used to monitor blood pressure at the target location, for example by connecting an arterial pressure monitoring device in fluid communication with a lumen 68 of the outer sheath 52. The port 58 can be also or alternatively be used to inject contrast agent, dye, thrombolytic agents such as tissue plasminogen activator (t-PA), etc. The slider 56 may not interact with the inner member 64 such that the inner member 64 is longitudinally movable independent of the filter assembly 66 and/or the outer sheath 52. The inner member translation control 60 can be used to longitudinally translate the inner member 64, for example before, after, and/or during deployment of the filter assembly 66. The inner member translation control 60 may comprise a slider in the handle 54 (e.g., separate from the slider 56).

The rotatable hemostasis valve control 62 can be used to reduce or minimize fluid loss through the protection device 40 during use. For example, when positioned in ascending aorta 26, the direction of blood flow with respect to the device 40 will be distal to proximal, so blood may be otherwise inclined to follow the pressure drop out of the device 40. The hemostasis valve control 62 is illustrated as being rotatable, but other arrangements are also possible (e.g., longitudinally displaceable). The hemostasis valve control 62 may be configured to fix relative positions of the outer sheath 52 and the filter assembly 66, for example as described with respect to the hemostasis valve in U.S. Pat. No. 8,876,796. The hemostasis valve 62 may comprise, for example, an elastomeric seal and hemostasis valve nut.

The distal portion 44 may comprise the outer sheath 52, a filter assembly 66 radially inward of the outer sheath 52 (in a delivery configuration), and optionally the inner member 64. The filter assembly 66 may be radially between the outer sheath 52 and the inner member 64 (e.g., radially inward of the outer sheath 52 and the inner member 64 radially inward of the filter assembly 66) in a delivery state or position.

The filter assembly 66 may include a support element or frame 46 and a filter element 50. The frame 46 may be a hoop-like structure configured to generally provide expansion support to the filter element 50 when the filter assembly 66 is in the expanded state (as shown in FIG. 1). In the expanded state, the filter element 50 may be configured to filter fluid (e.g., blood) flowing through the filter element 50 and to inhibit or prevent particles (e.g., embolic material) from flowing through the filter element 50 by capturing the particles in the filter element 50.

The frame 46 may be configured to engage or appose the inner walls of a lumen (e.g., blood vessel) in which the filter assembly 66 is expanded such that the filter assembly 66 is sealed against the wall of the vessel to ensure that most, if not all, blood flow exiting the aortic valve flows through the filter membrane 50. The frame 46 may comprise or be constructed of, for example, nickel titanium (e.g., nitinol), nickel titanium niobium, chromium cobalt (e.g., MP35N, 35NLT), copper aluminum nickel, iron manganese silicon, silver cadmium, gold cadmium, copper tin, copper zinc, copper zinc silicon, copper zinc aluminum, copper zinc tin, iron platinum, manganese copper, platinum alloys, cobalt nickel aluminum, cobalt nickel gallium, nickel iron gallium, titanium palladium, nickel manganese gallium, stainless steel, combinations thereof, and the like. The frame 46 may comprise a wire (e.g., having a round (e.g., circular, elliptical) or polygonal (e.g., square, rectangular) cross-section). For example, in some embodiments, the frame 46 comprises a straight piece of nitinol wire shape set into a circular or oblong hoop or hoop with one or two straight legs running longitudinally along or at an angle to a longitudinal axis of the filter assembly 66. At least one of the straight legs may be coupled to or form a part of the filter wire 48. The straight legs may be on a long side of the filter assembly 66 and/or on a short side of the filter assembly 66. In other embodiments, the frame 46 may be formed from laser cut nitinol (or other suitable material). The frame 46 may form a shape of an opening 70 of the filter assembly 66. The opening 70 may be circular, elliptical, or any shape that can appropriately appose sidewalls of a vessel such as the ascending aorta 26, aortic arch 30, innominate artery 12, etc. The filter assembly 66 may have a generally distally-facing opening 70. In other embodiments, the opening 70 may be proximally facing. The orientation (e.g., proximal facing or distal facing) of the opening 70 relative to the system 40 may vary depending on where the access incision is located.

The frame 46 may take other shapes as desired. In some embodiments, the frame 46 may take the general shape of an expandable or self-expanding stent frame. For example, the frame 46 may include a proximal hoop and a distal hoop. The proximal and distal hoops may be interconnected with one or more generally longitudinally extending struts. Some illustrative frames are described in commonly assigned U.S. Pat. No. 9,566,144, the entirety of which is hereby incorporated by reference.

The frame 46 may include a radiopaque marker such as a small coil wrapped around or coupled to the hoop to aid in visualization under fluoroscopy. In some embodiments, the frame 46 may be formed from or coated with a radiopaque material, such as tantalum, platinum iridium or other suitable material, in order to make the hoop visible under fluoroscopy. In some embodiments, the frame may comprise a shape other than a hoop, for example, a spiral. In some embodiments, the filter assembly 66 may not include or be substantially free of a frame.

In some embodiments, the frame 46 and the filter element 50 form an oblique truncated cone having a non-uniform or unequal length around and along the length of the filter assembly 66. In such a configuration, along the lines of a windsock, the filter assembly 66 has a larger opening 70 (upstream) diameter and a reduced ending (downstream) diameter.

The filter element 50 may include pores configured to allow blood to flow through the filter element 50, but that are small enough to inhibit and/or prevent particles such as embolic material from passing through the filter element 50. The filter element 50 may comprise a filter membrane such as a polymer (e.g., polyurethane, polytetrafluoroethylene (PTFE)) film mounted to the frame 46. In some embodiments, the filter element 50 may be made of a nitinol mesh, a stainless steel mesh, a polymer mesh (e.g., polyether ether ketone (PEEK), or any other suitable material or construction. For example, the filter element 50 may be formed from a knitted or woven material. The filter element 50 may have a thickness between about 0.0001 inches (0.0025 mm) and about 0.03 inches (0.76 mm) (e.g., no more than about 0.0001 inches, about 0.001 inches, about 0.005 inches, about 0.01 inches, about 0.015 inches, about 0.02 inches, about 0.025 inches, about 0.03 inches, ranges between such values, etc.).

The filter element 50 may comprise a plurality of pores or holes or apertures extending through the film. The film may be formed by weaving or braiding filaments or membranes and the pores may be spaces between the filaments or membranes. The filaments or membranes may comprise the same material or may include other materials (e.g., polymers, non-polymer materials such as metal, alloys such as nitinol, stainless steel, etc.). The pores of the filter element 50 are configured to allow fluid (e.g., blood) to pass through the filter element 50 and to resist the passage of embolic material that is carried by the fluid. The pores can be circular, elliptical, square, triangular, or other geometric shapes. Certain shapes such as an equilateral triangular, squares, and slots may provide geometric advantage, for example restricting a part larger than an inscribed circle but providing an area for fluid flow nearly twice as large, making the shape more efficient in filtration verses fluid volume. The pores may be laser drilled into or through the filter element 50, although other methods are also possible (e.g., piercing with microneedles, loose braiding or weaving). The pores may have a lateral dimension (e.g., diameter) between about 10 micron (μm) and about 1 mm (e.g., no more than about 10 μm, about 50 μm, about 100 μm, about 150 μm, about 200 μm, about 250 μm, about 300 μm, about 400 μm, about 500 μm, about 750 μm, about 1 mm, ranges between such values, etc.). Other pore sizes are also possible, for example, depending on the desired minimum size of material to be captured.

The material of the filter element 50 may comprise a smooth and/or textured surface that is folded or contracted into the delivery state by tension or compression into a lumen. For example, the filter element 50 and the frame 46 may be collapsed within the lumen 68 of the outer tubular member 52 for delivery. A reinforcement fabric may be added to or embedded in the filter element 50 to accommodate stresses placed on the filter element 50 during compression. A reinforcement fabric may reduce the stretching that may occur during deployment and/or retraction of the filter assembly 66. The embedded fabric may promote a folding of the filter element 50 to facilitate capture of embolic debris and enable recapture of an elastomeric membrane. The reinforcement material could comprise, for example, a polymer and/or metal weave to add localized strength. The reinforcement material could be imbedded into the filter element 50 to reduce thickness. For example, imbedded reinforcement material could comprise a polyester weave mounted to a portion of the filter element 50 near the longitudinal elements of the frame 46 where tensile forces act upon the frame 46 and filter element 50 during deployment and retraction of the filter assembly 66 from/into the outer sheath 52.

In some cases, the filter assembly 66 may include a self-expanding filter assembly (e.g., comprising a superelastic material with stress-induced martensite due to confinement in the outer sheath 52). The filter assembly 66, or portions thereof, may comprise a shape-memory material configured to self-expand upon a temperature change (e.g., heating to body temperature). The filter assembly 66, or portions thereof, may comprise a shape-memory or superelastic frame (e.g., comprising a distal end hoop comprising nitinol) and a microporous material (e.g., comprising a polymer including laser-drilled holes) coupled to the frame, for example similar to the filter assemblies described in U.S. Pat. No. 8,876,796.

The filter assembly 66 may be coupled (e.g., crimped, welded, soldered, etc.) to a distal end of the deployment wire or filter wire 48 via a strut or wire, although this is not required. When both or all of the filter wire 48 and the strut are provided, the filter wire 48 and the strut may be coupled within outer sheath 52 proximal to the filter assembly 66 using a crimp mechanism. In other embodiments, the filter wire 48 and the strut may be a single unitary structure. The filter wire 48 and/or strut can comprise a rectangular ribbon, a round (e.g., circular, elliptical) filament, a portion of a hypotube, a braided structure (e.g., as described herein), combinations thereof, and the like. The filter wire 48 can be coupled to the handle 54 and/or a slider to provide differential longitudinal movement versus the outer sheath 52, as shown by the arrows 72, which can sheathe and unsheathe the filter assembly 66 from the outer sheath 52. As described herein, the filter assembly may be unsheathed through actuation of the outer sheath 52.

The filter assembly 66 in an expanded, unconstrained state may have a maximum diameter or effective diameter (e.g., if the mouth is in the shape of an ellipse) sized for the desired vessel and/or a patient in which it is to be deployed. For example, a filter assembly 66 deployed in the aorta 10 may have a larger diameter than a filter assembly 66 deployed in the innominate artery 12. Different diameters can allow treatment of a selection of subjects having different vessel sizes. The filter assembly 66 may have a maximum length selected to minimize a pressure drop caused by the filter assembly 66. For example, passing the blood through a filter may reduce the flow rate of the blood passing through the filter and thus introduce a pressure drop from the opening 70 of the filter assembly 66 to the tail of the filter assembly 66. The length of the filter element 50 may be selected to reduce or minimize the pressure drop across the filter assembly 66. It is further contemplated that different filter lengths can allow treatment of a selection of subjects having different vessel sizes.

It is contemplated that a filter assembly 66 configured to be positioned within the ascending aorta 26 may be larger than a filter assembly 66 configured to be positioned in the innominate artery 12. For example, the ascending aorta 26 may have a diameter in the range of about 30 to 40 mm while the innominate artery 12 may have a diameter in the range of about 9 to 15 mm. Thus, a filter assembly 66 configured to be positioned within the ascending aorta may require a larger diameter such that the filter assembly 66 is in apposition with the vessel wall. However, a larger filter assembly 66 may increase the risk that the filter frame or hoop 46 may fold back on itself due to the high flow of blood through the filter which creates a high load on the filter due to the pressure drop created when blood flows through the filter membrane 50. For example, the frame 46 may bend back toward the outer shaft 52. In some cases, stiffening the frame 46 at the point where the frame 46 transitions into and/or connects to the filter wire 48 may help prevent the filter assembly 66 from folding back on itself. Alternatively or additionally, the surface area of the filter element 50 may be increased by, for example, increasing a length of the filter element 50. This may increase the number of holes or pores in the filter element 50 thus allowing more blood to flow through which may in turn reduce the pressure on the filter element 50. Other methods of increasing the surface area (and hence the number or area of pores) of the filter element 50 may also be used to reduce the pressure drop. For example, pleats or folds of material may be added to the filter element 50 to increase the surface area.

In some methods of use, the filter assembly 66 may first be withdrawn into the outer sheath 52 to collapse the filter assembly 66. A guidewire 74 may be inserted into a lumen (not explicitly shown) of the inner member 64 through a proximal or distal end thereof. Positioning the guidewire 74 within a lumen of the inner member 64 may allow for free movement of the guidewire 74 and may prevent compression of the guidewire 74 when the filter assembly 66 is sheathed in the outer sheath 52. The filter system 40 is advanced into the subject through an incision made in the subject's right radial artery, or alternatively the right brachial artery. Alternatively, the filter system 40 may be advanced using left radial access. In a variety of medical procedures, a medical instrument is advanced through a subject's femoral artery, which is larger than the right radial artery. A delivery catheter used in femoral artery access procedures has a larger outer diameter than would be allowed in a filter system advanced through a radial artery. Additionally, in some uses the filter system 40 is advanced from the right radial artery into the aorta via the brachiocephalic trunk. The radial artery has the smallest diameter of the vessels through which the system is advanced. The radial artery therefore limits the size of the system that can be advanced into the subject when the radial artery is the access point. The outer diameters of the systems described herein, when advanced into the subject via a radial artery, are therefore smaller than the outer diameters of the guiding catheters (or sheaths) typically used when access is gained via a femoral artery.

When the guidewire is used, the system 40 may be advanced along with the guidewire 74, the guidewire 74 extending distally beyond a distal end of the system 40 to protect the vessel from damage from the tip of the outer sheath 52. The system 40 may be advanced until the distal end of the outer sheath 52 is positioned above or adjacent to the aortic valve (not explicitly shown). Alternatively, the guidewire 74 may be advanced to the desired location and the system 40 advanced over the guidewire 74 after the distal end of the guidewire 74 is positioned at the target location.

In certain anatomies, when the outer sheath 52 is advanced through the innominate artery 12 and into the aorta 10, the outer sheath 52 may tend to advance down the descending aorta 28 instead of the ascending aorta 26. In order to prevent this, or adjust for this, the outer sheath 52 may be made steerable so that the operator can guide the tip of the outer sheath 52 into the ascending aorta 26 and towards the aortic valve. In one embodiment, the outer sheath 52 may include a deflectable tip that is actuated by a pull-wire controlled at the device handle 54. Alternatively, or additionally, the outer sheath 52 may include a pre-shaped deflected tip (e.g., similar to the J-tip on a guidewire) that would allow the operator to rotate the outer sheath 52 to aim the outer sheath 52 tip down the ascending aorta 26. It is further contemplated that a pre-shaped tip may be straightened by inserting a semi-rigid or rigid stylet into a lumen in the outer sheath 52 (and/or inner member 64). This would allow the outer sheath 52 to be inserted while straight, and then when the tip of the outer sheath 52 is in the aorta 10, the stylet could be withdrawn, thus allowing the pre-shaped tip of the outer sheath 52 to deflect in the direction of the ascending aorta 26. Other methods of deflecting the tip may also be used

When placing the system 40, it may be desirable to avoid contact with the aortic valve to avoid damaging it. This may be accomplished by inserting a pigtail angiography catheter into the aortic arch through either a femoral access or through a left radial artery access, and injecting contrast so that the aortic valve may be located and imaged using fluoroscopy. However, it may be desired to not require the addition of femoral access for an angiography catheter given that femoral access is not required for all index procedure catheters, and femoral access is associated with increased access site vascular complications. It is contemplated that an angiography catheter (in some cases, a 4 Fr angiography catheter) may be advanced through an inner lumen 68 of the outer sheath 52 and/or the inner member 64 and into the filter assembly 66 after deployment of the filter assembly 66. Contrast may then be injected through the angiography catheter. In another embodiment, contrast may be injected directly through the guidewire lumen of the inner member 64 or through the inner lumen of the outer sheath 52. In yet another embodiment, contrast may not be used and the filter assembly 66 may be placed with imaging using transesophageal echocardiography (TEE).

Once the system 40 is in a desired position, the guidewire 74 may then be proximally retracted. The system 40 may be delivered to the ascending aorta 26 in a delivery configuration. The system's delivery configuration generally refers to the configuration where the filter assembly 66 is in a collapsed configuration within the system 40 (e.g., within the outer sheath 52). The outer sheath 52 is retracted proximally to allow the filter frame 46 to expand to an expanded configuration against the wall of the ascending aorta 26 upstream of the ostium of the innominate artery 12, as is shown in FIG. 1. The filter element 50 is secured either directly or indirectly to the support frame 46 and is therefore reconfigured to the configuration shown in FIG. 1 upon deployment of the frame 46. Alternatively, or additionally, the filter assembly 66 may be distally advanced from the outer sheath 52 through distal actuation of the filter wire 48. Once expanded, the filter assembly 66 filters blood traveling through the ascending aorta 26, and therefore filters blood traveling into innominate artery 12, the right common carotid artery 18, the right vertebral artery 20, the left common carotid artery 14, the left subclavian artery 16, the left vertebral artery 24, and the descending aorta 28. The expanded filter assembly 66 is therefore in position to prevent foreign particles from traveling into all four cerebral arteries 14, 18, 20, 24, and into the cerebral vasculature. It is contemplated that the inner member 64 may be removable following deployment of the filter assembly 66, although this is not required.

The filter assembly 66 may be re-sheathed or positioned within the outer sheath 52 to facilitate repositioning of the filter assembly to optimize the placement of the filter assembly 66. To re-sheathe the filter assembly 66, it is contemplated that the outer sheath 52 may be distally advanced over the filter assembly 66, the filter assembly 66 proximally retracted into the outer sheath 52 via the filter wire 48, or combinations thereof. The system 40 may then be repositioned and the filter assembly 66 redeployed. The filter assembly 66 may be re-sheathed, repositioned, and redeployed as many times as desired to optimize the placement of the filter assembly 66.

Once the filter assembly 66 has been deployed, the index procedure (e.g., LAOO, TMVR, ablation, etc.) may then be performed. Following the completion of the index procedure, the filter assembly 66 may be re-sheathed (e.g., collapsed within the outer sheath 52) and the system 40 removed, with any captured debris removed along with the filter assembly 66.

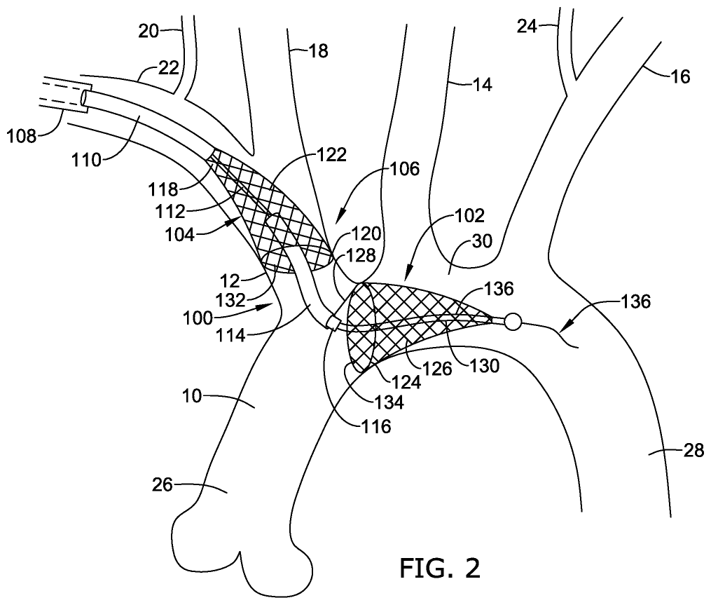

FIG. 2 illustrates an alternative protection device 100 including a distal filter assembly 102 and a proximal filter assembly 104 and positioned using a right radial access incision. The protection device 100 may include a distal end region 106 and a proximal end region (not explicitly shown). The proximal end region may be configured to be held and manipulated by a user such as a surgeon. The distal end region 106 may be configured to be positioned at a target location such as, but not limited to, the innominate artery 12 and/or the aortic arch 30. When the distal end region 106 is so deployed, blood is filtered prior to entering the left common carotid artery 14, the left subclavian artery 16, the left vertebral artery 24, the right common carotid artery 18, and the right vertebral artery 20.

The proximal end region may be similar in form and function to the proximal end region 42 described herein. While not explicitly shown, the proximal end region may include a handle, a control such as a slider, an outer sheath, a port, an inner member translation control such as a knob, and hemostasis valve control such as a knob. In some embodiments, the proximal end region may include fewer or more control elements than those illustrated and described with respect to FIG. 1. The proximal end region may also include an inner member radially inward of the outer sheath 108. While not explicitly shown, the proximal end region may also include a filter wire radially inward of the outer sheath (and sometimes radially outward of the inner member). Some illustrative filter wires are described in commonly assigned U.S. Pat. No. 9,566,144, the entirety of which is hereby incorporated by reference.

The distal end region 106 may include a first or distal filter assembly 102 configured to be deployed within the aortic arch 30 (upstream of the ostium of the left common carotid artery 14) and a second or proximal filter assembly 104 configured to deployed within the innominate artery 12. The distal end region 106 may further include a proximal (or outer) sheath 108, a proximal shaft 110 coupled to an expandable proximal filter assembly 104, a distal shaft 112 coupled to a distal articulatable sheath 114, a distal filter assembly 102, and guiding member 116.

The proximal shaft 110 is co-axial with proximal sheath 108, and a proximal region 118 of proximal filter assembly 104 is secured to proximal shaft 110. In its collapsed configuration (not explicitly shown), the proximal filter assembly 104 may be disposed within proximal sheath 108 and is disposed distally relative to the proximal shaft 110. The proximal sheath 108 may be axially (e.g., distally and proximally) movable relative to proximal shaft 110 and the proximal filter assembly 104. The system 100 may also include a distal sheath 114 secured to a distal region of the distal shaft 112. The distal shaft 112 may be co-axial with the proximal shaft 110 and the proximal sheath 108. The distal sheath 114 and distal shaft 112 may be secured to one another and axially movable relative to the proximal sheath 108, the proximal shaft 110, and the proximal filter assembly 104. The system 100 may also include a distal filter assembly 102 carried by the guiding member 116. While not explicitly shown, the distal filter assembly 102 may be maintained in a collapsed configuration within the distal sheath 114. The guiding member 116 may be coaxial with the distal sheath 114 and the distal shaft 112 as well as the proximal sheath 108 and the proximal shaft 110. The guiding member 116 may be axially movable relative to the distal sheath 114 and the distal shaft 112 as well as the proximal sheath 108 and the proximal shaft 110. The proximal sheath 108, the distal sheath 114, and the guiding member 116 may each be adapted to be independently moved axially relative to one other. That is, the proximal sheath 108, the distal sheath 114, and the guiding member 116 are adapted for independent axial translation relative to each of the other two components. It is contemplated that a handle, which may be similar in form and function to the handle 54 described herein, may include control elements (such as, but not limited to, slides, switches, buttons, dials, etc.) configured to individually actuate the proximal sheath 108, the distal sheath 114, and the guiding member 116.

The proximal filter assembly 104 may include a support element or frame 120 and a filter element 122. Similarly, the distal filter assembly 102 includes support element 124 and a filter element 126. The frames 120, 124 may generally provide expansion support to the filter elements 122, 126 in the expanded state. In the expanded state, the filter elements 122, 126 are configured to filter fluid (e.g., blood) flowing through the filter elements 122, 126 and to inhibit or prevent particles (e.g., embolic material) from flowing through the filter elements 122, 126 by capturing the particles in the filter elements 122, 126. The frames 120, 124 are configured to engage or appose the inner walls of a lumen (e.g., blood vessel) in which the filter assembly 102, 104 is expanded. The frames 120, 124 may be similar in form and function the frame 46 described herein and the filter elements 122, 126 may be similar in form and function to the filter element 50 described herein. For example, in some embodiments, the frames 120, 124 may comprise a straight piece of nitinol wire shape set into a circular or oblong hoop or hoops with one or two straight legs running longitudinally along or at an angle to a longitudinal axis of the filter assembly 102, 104. At least one of the straight legs may be coupled to a filter wire 130 or a strut 128, as shown with respect to the distal filter assembly 102. The straight legs may be on a long side of the filter assembly 102, 104 and/or on a short side of the filter assembly 102, 104. The frames 120, 124 may form a shape of an opening 132, 134 of the filter assembly 102, 104. The opening 132, 134 may be circular, elliptical, or any shape that can appropriately appose sidewalls of a vessel. The distal filter assembly 102 may have a generally proximally-facing opening 134. The proximal filter assembly 104 may have a generally distally-facing opening 132. The orientation of the opening 132, 134 may vary depending on where the access incision is located. For example, as shown in FIG. 2, the proximal filter assembly 104 has a generally distally-facing opening 132, and the distal filter assembly 102 has a generally proximally-facing opening 134 relative to the system 100 for introduction via the right side of the body. It is contemplated that the configuration of the openings 132, 134 may be reversed for introduction via the left side of the body. The filter assemblies 102, 104 can be thought of as facing opposite directions.

In some cases, the filter assembly 102, 104 may include a self-expanding filter assembly (e.g., comprising a superelastic material with stress-induced martensite due to confinement in the outer sheath 108 and/or the distal sheath 114). The filter assembly 102, 104 may comprise a shape-memory material configured to self-expand upon a temperature change (e.g., heating to body temperature). The filter assembly 102, 104 may comprise a shape-memory or superelastic frame (e.g., comprising a distal end hoop comprising nitinol) and a microporous material (e.g., comprising a polymer including laser-drilled holes) coupled to the frame, for example similar to the filter assemblies described in U.S. Pat. No. 8,876,796.

The distal filter assembly 102 may be coupled (e.g., crimped, welded, soldered, etc.) to a distal end of a deployment wire or filter wire 130 via a strut or wire 128, although this is not required. In some embodiments, the proximal filter assembly 104 may also be coupled to a filter wire or strut (not explicitly shown). When both or all of the filter wire 130 and the strut 128 are provided, the filter wire 130 and the strut 128 may be coupled within the guiding member 116 proximal to the filter assembly 102 using a crimp mechanism. In other embodiments, the filter wire 130 and the strut 128 may be a single unitary structure. The filter wire 130 and/or strut 128 can comprise a rectangular ribbon, a round (e.g., circular, elliptical) filament, a portion of a hypotube, a braided structure (e.g., as described herein), combinations thereof, and the like. The filter wire 130 can be coupled to the handle (not explicitly shown) and/or a slider to provide differential longitudinal movement versus the outer sheath 108, which can sheathe and unsheathe the distal filter assembly 102 from the distal sheath 114. Similarly, the proximal filter assembly 104 may be unsheathe through actuation of a mechanism on the handle or through movement of the handle itself.

The filter assemblies 102, 104 in an expanded, unconstrained state may have a maximum diameter or effective diameter (e.g., if the mouth is in the shape of an ellipse). The diameter can be between about 1 mm and about 40 mm, or more. Other diameters or other types of lateral dimensions are also possible. Different diameters can allow treatment of a selection of subjects having different vessel sizes. The filter assemblies 102, 104 may have a maximum length. The length can be between about 7 mm and about 50 mm, or more. Other lengths are also possible, for example based on the diameter or effective diameter. For example, the length of the filter assembly 102, 104 may increase as the diameter increases, and the length of the filter assembly 102, 104 may decrease as the diameter decreases. A distance from an apex of the mouth of the filter assembly 102, 104 to an elbow in the frame may be about 35 mm. Different lengths can allow treatment of a selection of subjects having different vessel sizes.

As described in more detail herein, the distal sheath 114 may be adapted to be steered, or bent, relative to the proximal sheath 108 and the proximal filter assembly 104. As the distal sheath 114 is steered, the relative directions in which the openings face will be adjusted. Regardless of the degree to which the distal sheath 114 is steered, the filter assemblies 102, 104 are still considered to having openings facing opposite directions. For example, the distal sheath 114 could be steered to have an approximately 90 degree bend, in which case the filter assemblies 102, 104 would have openings 132, 134 facing at generally orthogonal angles, as shown in FIG. 2. The directions of the filter openings 132, 134 are therefore described if the system were to assume a substantially straightened configuration (not explicitly shown). The proximal filter element 122 may taper down in the proximal direction from support element 120, while the distal filter element 126 may taper down in the distal direction from support element 124. A fluid, such as blood, flows through the opening and passes through the pores in the filter elements 122, 126, while the filter elements 122, 126 are adapted to trap foreign particles therein and prevent their passage to a location downstream of the filter assemblies.

The filter assemblies 102, 104 may be secured to separate system components. For example, the proximal filter assembly 104 is secured to the proximal shaft 110, while the distal filter assembly 102 is secured to guiding member 116. In FIG. 2, the filter assemblies 102, 104 are secured to independently actuatable components. This may allow the filter assemblies 102, 104 to be independently positioned and controlled. Additionally, the filter assemblies 102, 104 may be collapsed within two different tubular members in their collapsed configurations. For example, the proximal filter assembly 104 is collapsed within proximal sheath 108, while the distal filter assembly 102 is collapsed within distal sheath 114. In the system's delivery configuration, the filter assemblies 102, 104 are axially-spaced from one another. For example, in FIG. 2, the distal filter assembly 102 is distally-spaced relative to proximal filter assembly 104. However, in an alternative embodiment, the filter assemblies 102, 104 may be positioned such that a first filter is located within a second filter.

In some embodiments, the distal sheath 114 and the proximal sheath 108 have substantially the same outer diameter. When the filter assemblies 102, 104 are collapsed within the respective sheaths 114, 108, the sheath portion of the system 100 therefore has a substantially constant outer diameter, which can ease the delivery of the system 100 through the patient's body and increase the safety of the delivery. The distal and proximal sheaths 114, 108 may have substantially the same outer diameter, both of which have larger outer diameters than the proximal shaft 110. The proximal shaft 110 may have a larger outer diameter than the distal shaft 112, wherein the distal shaft 112 is disposed within the proximal shaft 110. The guiding member 116 may have a smaller diameter than the distal shaft 112. In some embodiments, the proximal and distal sheaths 108, 114 have an outer diameter between about 3 French (F) and 14 F. In certain embodiments, the outer diameter is between about 4 F and 8 F. In still other embodiments, the outer diameter is between 4 F and 6 F. In some embodiments, the sheaths 108, 114 have different outer diameters. For example, the proximal sheath 108 can have a size of 6 F, while the distal sheath 114 has a size of 5 F. In an alternate embodiment the proximal sheath 108 is about 5 F and the distal sheath 114 is about 4 F. These are just examples and are not intended to limit the sheaths 108, 114 to a particular size. A distal sheath 114 with a smaller outer diameter than the proximal sheath 108 may reduce the delivery profile of the system 100 and can ease delivery.

In some methods of use, the filter system 100 is advanced into the subject through an incision made in the subject's right radial artery, or alternatively the right brachial artery. In a variety of medical procedures, a medical instrument is advanced through a subject's femoral artery, which is larger than the right radial artery. A delivery catheter used in femoral artery access procedures may have a larger outer diameter than would be allowed in a filter system advanced through a radial artery. Additionally, in some uses the filter system is advanced from the right radial artery into the aorta via the brachiocephalic trunk. The radial artery has the smallest diameter of the vessels through which the system is advanced. The radial artery therefore limits the size of the system that can be advanced into the subject when the radial artery is the access point. The outer diameters of the systems described herein, when advanced into the subject via a radial artery, are therefore smaller than the outer diameters of the guiding catheters (or sheaths) typically used when access is gained via a femoral artery. In some embodiments, the system 100 may be advanced over a guidewire 136, although this is not required.

The system 100 may be delivered to the aortic arch 30 and the innominate artery 12 in a delivery configuration. The system's delivery configuration generally refers to the configuration where both filter assemblies 102, 104 are in collapsed configurations within the system (e.g., within the distal and proximal sheaths 114, 108). The distal articulating sheath 114 may be independently movable with 3 degrees of freedom relative to the proximal sheath 108 and proximal filter assembly 104. In some embodiments, the proximal sheath 108 and the distal sheath 114 may be releasably coupled together. For example, the proximal sheath 108 can be coupled to the distal sheath 114 using an interference fit, a friction fit, a spline fitting, end to end butt fit, or any other type of suitable coupling between the two sheaths 108, 114. When coupled together, the components move as a unit. For example, the proximal sheath 108, the proximal shaft 110, the proximal filter assembly 104, the distal shaft 112, and the distal filter assembly 102 will rotate and translate axially (in the proximal or distal direction) as a unit. When the proximal sheath 108 is retracted to allow the proximal filter assembly 104 to expand, the distal sheath 114 can be independently rotated, steered, or translated axially (either in the proximal direction or distal direction). The distal sheath 114 therefore has 3 independent degrees of freedom: axial translation, rotation, and steering. The adaptation to have 3 independent degrees of freedom is advantageous when positioning the distal sheath 114 in a target location, details of which are described herein.

The system 100 is advanced into the subject's right radial artery through an incision in the right arm, or alternately through the right brachial artery. The system is advanced through the right subclavian artery 22 and into the brachiocephalic or innominate artery 12, and a portion of the system is positioned within the aortic arch 30. The proximal sheath 108 is retracted proximally to allow proximal filter support element 120 to expand to an expanded configuration against the wall of the innominate artery 12, as is shown in FIG. 2. The proximal filter element 122 is secured either directly or indirectly to support element 120 and is therefore reconfigured to the configuration shown in FIG. 2. The position of distal sheath 114 can be substantially maintained while proximal sheath 108 is retracted proximally. Once expanded, the proximal filter assembly 104 filters blood traveling through the innominate artery 12, and therefore filters blood traveling into the right common carotid artery 18 and the right vertebral artery 20. The expanded proximal filter assembly 104 is therefore in position to prevent foreign particles from traveling into the right common carotid artery 18 and the right vertebral artery 20 and into the cerebral vasculature.

The distal sheath 114 is then steered, or bent, and a distal end of the distal sheath 114 is positioned within the aortic arch 30 such that the distal filter support element 124 is configured to be deployed upstream of the ostium of the left common carotid artery 14 (and downstream of the ostium of the innominate artery 12). The guiding member 116 is thereafter advanced distally relative to distal sheath 114, allowing the distal support element 124 to expand from a collapsed configuration to a deployed configuration against the wall of the aortic arch 30, as shown in FIG. 2. The distal filter element 126 is also reconfigured into the configuration shown in FIG. 2. Once expanded, the distal filter assembly 102 filters blood traveling through the aortic arch 30 and hence the left common carotid artery 14 and left subclavian artery 16. The distal filter assembly 102 is therefore in position to trap foreign particles and prevent them from traveling into the cerebral vasculature. In some embodiments, the distal filter assembly 102 may be deployed prior to the deployment of the proximal filter assembly 104.

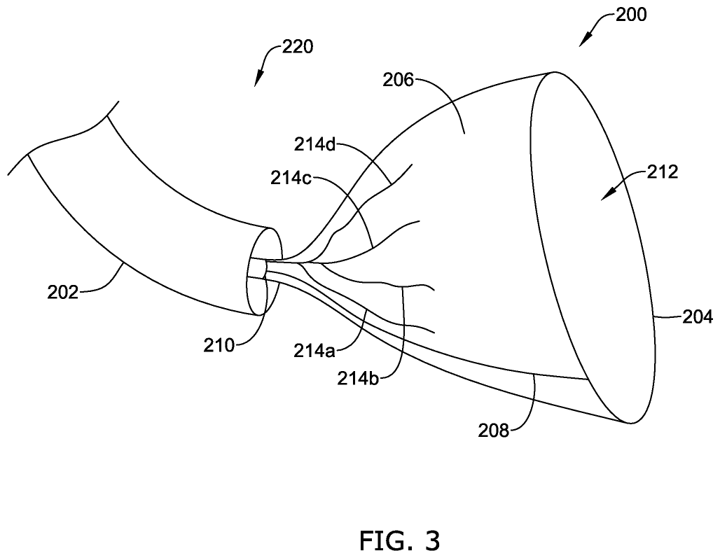

FIG. 3 is a partial perspective view of another illustrative filter assembly 200. The filter assembly 200 may be configured to be advanced to the target location within an outer sheath 202 that may be similar in form and function to the outer sheath 52 described with respect to FIG. 1. The filter assembly 200 may include a support element or frame 204 and a filter element 206. The frame 204 may be a hoop-like structure configured to generally provide expansion support to the filter element 206 in the expanded state. In the expanded state, the filter element 206 may be configured to filter fluid (e.g., blood) flowing through the filter element 206 and to inhibit or prevent particles (e.g., embolic material) from flowing through the filter element 206 by capturing the particles in the filter element 206.

The frame 204 may be similar in form and function the frame 46 described herein and the filter element 206 may be similar in form and function to the filter element 50 described herein. For example, in some embodiments, the frame 204 comprises a straight piece of nitinol wire shape set into a circular or oblong hoop or hoop with one or two straight legs 208 running longitudinally along or at an angle to a longitudinal axis of the filter assembly 200. At least one of the straight legs may be coupled to a filter wire 210 or a strut. The straight legs may be on a long side of the filter assembly 200 and/or on a short side of the filter assembly 200. The frame 204 may form a shape of an opening 212 of the filter assembly 200. The opening 212 may be circular, elliptical, or any shape that can appropriately appose sidewalls of a vessel. The filter assembly 200 may have a generally distally-facing opening 212. In other embodiments, the opening 212 may be proximally facing. The orientation of the opening 212 may vary depending on where the access incision is located and/or the vessel in which the filter assembly 200 is to be positioned.

In some cases, the filter assembly 200 may include a self-expanding filter assembly (e.g., comprising a superelastic material with stress-induced martensite due to confinement in the outer sheath 202). The filter assembly 200 may comprise a shape-memory material configured to self-expand upon a temperature change (e.g., heating to body temperature). The filter assembly 200 may comprise a shape-memory or superelastic frame (e.g., comprising a distal end hoop comprising nitinol) and a microporous material (e.g., comprising a polymer including laser-drilled holes) coupled to the frame, for example similar to the filter assemblies described in U.S. Pat. No. 8,876,796.

The filter assembly 200 may be coupled (e.g., crimped, welded, soldered, etc.) to a distal end of a deployment wire or filter wire 210 via a strut or wire, such as, but not limited to the leg 208, although this is not required. When both or all of the filter wire 210 and the strut 208 are provided, the filter wire 210 and the strut may be coupled proximal to the filter assembly 200 using a crimp mechanism. In other embodiments, the filter wire 210 and the strut 208 may be a single unitary structure. The filter wire 210 and/or strut 208 can comprise a rectangular ribbon, a round (e.g., circular, elliptical) filament, a portion of a hypotube, a braided structure (e.g., as described herein), combinations thereof, and the like. The filter wire 210 can be coupled to the handle (not explicitly shown) and/or a slider to provide differential longitudinal movement versus the outer sheath 202, which can sheathe and unsheathe the filter assembly 200 from the outer sheath 202.

It is contemplated that the filter assembly 200 may further include one or more support elements or tines 214a, 214b, 214c, 214d (collectively, 214). The one or more tines 214 may extend distally from the filter wire 210 towards the filter frame 204. For a proximally facing filter, the one or more tines 214 may extend proximally from a distal end of the filter towards the filter frame. It is contemplated that the one or more tines 214 may extend an entire length of the filter assembly 200 from the filter wire 210 to the filter frame 204 or less than an entire length between the filter wire 210 and the filter frame 204. In some embodiments, the one or more tines 214 may extend 99% or less, 90% or less, 70% or less, 50% or less, 30% or less of the length between the filter wire 210 and the filter frame 204. These are just some examples. The one or more tines 214 may extend over any length between the filter wire 210 and the filter frame 204, as desired. In some embodiments, the tines 214 may be uniformly spaced at approximately equal intervals about the filter membrane 206. In other embodiments, the tines 214 may be eccentrically positioned, as desired. It is further contemplated that the filter assembly 200 may include any number of tines 214, such as, but not limited to, zero, one, two, three, four, five, six, or more. The tines 214 may be formed from a material that is more rigid than the filter element 206 such that the tines 214 provide additional structural support to the filter assembly 200.

The delivery of the filter assembly 200 may be similar to the delivery of the filter assembly 66 described herein. In some methods of use, the filter assembly 200 may first be withdrawn into the outer sheath 202 to collapse (not explicitly shown) the filter assembly 200. The outer sheath 202 may be advanced to the target location with or without a guidewire, as desired. The filter system 220 (e.g., filter assembly 200, outer sheath 202, and other delivery components) may be advanced into the subject through an incision made in the subject's right radial artery, or alternatively the right brachial artery. However, the system 220 may be advanced using an access location desired, including, but not limited to left radial access, left brachial access, femoral access, etc.

When placing the system 220, it may be desirable to avoid contact with the aortic valve to avoid damaging it. This may be accomplished by inserting a pigtail angiography catheter into the aortic arch through either a femoral access or through a left radial artery access, and injecting contrast so that the aortic valve may be located and imaged using fluoroscopy. However, it may be desired to not require the addition of femoral access for an angiography catheter given that femoral access is not required for all index procedure catheters, and femoral access is associated with increased access site vascular complications. It is contemplated that an angiography catheter (in some cases, a 4 Fr angiography catheter) may be advanced through an inner lumen of the outer sheath 202 and into the filter assembly 200 before or after deployment of the filter assembly 200. Contrast may then be injected through the angiography catheter. In another embodiment, contrast may be injected directly through the guidewire lumen of an inner member (if so provided) or through the inner lumen of the outer sheath 202 if the inner member is removed. In yet another embodiment, contrast may not be used and the filter assembly 200 may be placed with imaging using transesophageal echocardiography (TEE).

Once the system 220 is in a desired position, the guidewire (if used) may then be proximally retracted. The system 220 may be delivered to the ascending aorta (see, for example, FIG. 1) in a delivery configuration. The system's delivery configuration generally refers to the configuration where the filter assembly 200 is in a collapsed configuration within the outer sheath 202. The outer sheath 202 is retracted proximally to allow filter frame 204 to expand to an expanded configuration against the wall of the ascending aorta upstream of the ostium of the innominate artery. The filter element 206 is secured either directly or indirectly to support frame 204 and is therefore reconfigured to the configuration shown in FIG. 3. Alternatively, or additionally, the filter assembly 200 may be distally advanced from the outer sheath 202 through distal actuation of the filter wire 210. Once expanded, the filter assembly 200 filters blood traveling through the ascending aorta, and therefore filters blood traveling into innominate artery, the right common carotid artery, the right vertebral artery 20, the left common carotid artery 14, the left subclavian artery 16, the left vertebral artery, and the descending aorta. The expanded filter assembly 200 is therefore in position to prevent foreign particles from traveling into all four cerebral arteries, and into the cerebral vasculature.

The filter assembly 200 may be re-sheathed or positioned within the outer sheath 202 to facilitate repositioning of the filter assembly to optimize the placement of the filter assembly 200. To re-sheathe the filter assembly 200, it is contemplated that the outer sheath 202 may be distally advanced over the filter assembly 200, the filter assembly 200 proximally retracted into the outer sheath via the filter wire 210, or combinations thereof. The system 220 may then be repositioned and the filter assembly 200 redeployed. The filter assembly 200 may be re-sheathed, repositioned, and redeployed as many times as desired to optimize the placement of the filter assembly 200.

Once the filter assembly 200 has been deployed, the index procedure (e.g., LAOO, TMVR, ablation, etc.) may then be performed. Following the completion of the index procedure, the filter assembly 200 may be sheathed (e.g., collapsed within the outer sheath 202) and the system 220 removed, with any captured debris removed along with the filter assembly 200.

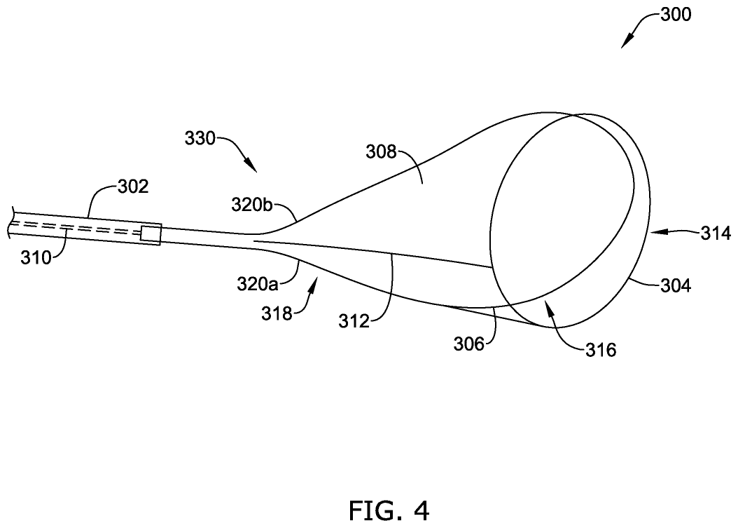

FIG. 4 is a partial perspective view of another illustrative filter assembly 300. The filter assembly 300 may be configured to be advanced to a target location within an outer sheath 302 that may be similar in form and function to the outer sheath 52 described with respect to FIG. 1. The filter assembly 300 may include a support element or frame 304 and a filter element 308. The frame 304 may be a hoop-like structure configured to generally provide expansion support to the filter element 308 in the expanded state. In the expanded state, the filter element 308 may be configured to filter fluid (e.g., blood) flowing through the filter element 308 and to inhibit or prevent particles (e.g., embolic material) from flowing through the filter element 308 by capturing the particles in the filter element 308.

The frame 304 may be similar in form and function the frame 46 described herein and the filter element 308 may be similar in form and function to the filter element 50 described herein. For example, in some embodiments, the frame 304 comprises a straight piece of nitinol wire shape set into a circular or oblong hoop or hoop with one or two straight legs 312 running longitudinally along or at an angle to a longitudinal axis of the filter assembly 300, although this is not required. At least one of the straight legs 312 may be coupled to a filter wire 310 or a strut. The straight legs 312 may be on a long side of the filter assembly 300 and/or on a short side of the filter assembly 300. The frame 304 may form a shape of an opening 314 of the filter assembly 300. The opening 314 may be circular, elliptical, or any shape that can appropriately appose sidewalls of a vessel. The filter assembly 300 may have a generally distally-facing opening 314. In other embodiments, the opening 314 may be proximally facing. The orientation of the opening 314 may vary depending on where the access incision is located and/or the vessel in which the filter assembly 300 is to be positioned.

In some embodiments, the filter assembly 300 may include a second, or an additional hoop-like support structure 306. The support structure 306 may have a curved distal end region 316 which extends about a portion of the circumference of the filter assembly 300 and a generally longitudinally extending proximal portion 318. The proximal portion 318 may include pair of legs 320a, 320b (collectively, 320). A proximal portion of the legs 320 may be secured to the filter wire 310 while the distal ends of the legs 320 are interconnected by the distal portion 316 of the support structure 306. The support structure 306 may be positioned proximal to the filter frame 304 to stiffen the filter element 308 and to ensure good expansion and apposition of the filter assembly 300. However, other arrangements of the support structure 306 and/or the filter frame 304 are also contemplated. For example, in one embodiment, the support structure 306 may be positioned distal to the filter frame 304. Alternatively, or additionally, the support structure 306 may have a helical or spring-like shape.

In some cases, the filter assembly 300 may include a self-expanding filter assembly (e.g., comprising a superelastic material with stress-induced martensite due to confinement in the outer sheath 302). The filter assembly 300 may comprise a shape-memory material configured to self-expand upon a temperature change (e.g., heating to body temperature). The filter assembly 300 may comprise a shape-memory or superelastic frame (e.g., comprising a distal end hoop comprising nitinol) and a microporous material (e.g., comprising a polymer including laser-drilled holes) coupled to the frame, for example similar to the filter assemblies described in U.S. Pat. No. 8,876,796.

The filter assembly 300 may be coupled (e.g., crimped, welded, soldered, etc.) to a distal end of a deployment wire or filter wire 310 via a strut or wire, such as, but not limited to the leg 312 and/or legs 320, although this is not required. When both or all of the filter wire 310 and the strut are provided, the filter wire 310 and the strut may be coupled proximal to the filter assembly 300 using a crimp mechanism. In other embodiments, the filter wire 310 and the strut (or leg 312) may be a single unitary structure. The filter wire 310 and/or strut can comprise a rectangular ribbon, a round (e.g., circular, elliptical) filament, a portion of a hypotube, a braided structure (e.g., as described herein), combinations thereof, and the like. The filter wire 310 can be coupled to the handle (not explicitly shown) and/or a slider to provide differential longitudinal movement versus the outer sheath 302, which can sheathe and unsheathe the filter assembly 300 from the outer sheath 302.

The delivery of the filter assembly 300 may be similar to the delivery of the filter assembly 66 described herein. In some methods of use, the filter assembly 300 may first be withdrawn into the outer sheath 302 to collapse the filter assembly 300 (not explicitly shown). The outer sheath 302 may be advanced to the target location with or without a guidewire, as desired. The filter system 330 (e.g., filter assembly 300, outer sheath 302, and other delivery components) may be advanced into the subject through an incision made in the subject's right radial artery, or alternatively the right brachial artery. However, the system 330 may be advanced using an access location desired, including, but not limited to left radial access, left brachial access, femoral access, etc.

When placing the system 330, it may be desirable to avoid contact with the aortic valve to avoid damaging it. This may be accomplished by inserting a pigtail angiography catheter into the aortic arch through either a femoral access or through a left radial artery access, and injecting contrast so that the aortic valve may be located and imaged using fluoroscopy. However, it may be desired to not require the addition of femoral access for an angiography catheter given that femoral access is not required for all index procedure catheters, and femoral access is associated with increased access site vascular complications. It is contemplated that an angiography catheter (in some cases, a 4 Fr angiography catheter) may be advanced through an inner lumen of the outer sheath 302 and into the filter assembly 300 before or after deployment of the filter assembly 300. Contrast may then be injected through the angiography catheter. In another embodiment, contrast may be injected directly through the guidewire lumen of an inner member (if so provided) or through the inner lumen of the outer sheath 302 if the inner member is removed. In yet another embodiment, contrast may not be used and the filter assembly 300 may be placed with imaging using transesophageal echocardiography (TEE).

Once the system 330 is in a desired position, the guidewire (if used) may then be proximally retracted. The system 330 may be delivered to the ascending aorta (see, for example, FIG. 1) in a delivery configuration. The system's delivery configuration generally refers to the configuration where the filter assembly 300 is in a collapsed configuration within the outer sheath 302. The outer sheath 302 is retracted proximally to allow the filter frame 304 to expand to an expanded configuration against the wall of the ascending aorta upstream of the ostium of the innominate artery. The filter element 308 is secured either directly or indirectly to support frame 304 and is therefore reconfigured to the configuration shown in FIG. 4. Alternatively, or additionally, the filter assembly 300 may be distally advanced from the outer sheath 302 through distal actuation of the filter wire 310. Once expanded, the filter assembly 300 filters blood traveling through the ascending aorta, and therefore filters blood traveling into innominate artery, the right common carotid artery 18, the right vertebral artery, the left common carotid artery, the left subclavian artery, the left vertebral artery, and the descending aorta. The expanded filter assembly 300 is therefore in position to prevent foreign particles from traveling into all four cerebral arteries, and into the cerebral vasculature.

The filter assembly 300 may be re-sheathed or positioned within the outer sheath 302 to facilitate repositioning of the filter assembly to optimize the placement of the filter assembly 300. To re-sheathe the filter assembly 300, it is contemplated that the outer sheath 302 may be distally advanced over the filter assembly 300, the filter assembly 300 proximally retracted into the outer sheath 302 via the filter wire 310, or combinations thereof. The system 330 may then be repositioned and the filter assembly 300 redeployed. The filter assembly 300 may be re-sheathed, repositioned, and redeployed as many times as desired to optimize the placement of the filter assembly 300.

Once the filter assembly 300 has been deployed, the index procedure (e.g., LAOO, TMVR, ablation, etc.) may then be performed. Following the completion of the index procedure, the filter assembly 300 may be sheathed (e.g., collapsed within the outer sheath 302) and the system 330 removed, with any captured debris removed along with the filter assembly 300.

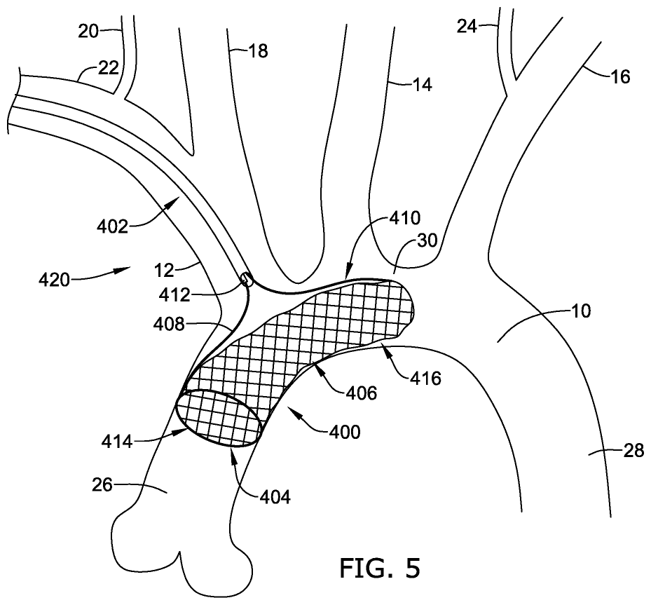

FIG. 5 is a schematic view of a portion of an aorta 10 including a filter assembly 400. The filter assembly 400 may be configured to be advanced to the target location within an outer sheath 402 that may be similar in form and function to the outer sheath 52 described with respect to FIG. 1. The filter assembly 400 may include a support element or frame 404 and a filter element 406. The frame 404 may be a hoop-like structure configured to generally provide expansion support to the filter element 406 in the expanded state. In the expanded state, the filter element 406 may be configured to filter fluid (e.g., blood) flowing through the filter element 406 and to inhibit or prevent particles (e.g., embolic material) from flowing through the filter element 406 by capturing the particles in the filter element 406.

The frame 404 may be similar in form and function the frame 46 described herein and the filter element 406 may be similar in form and function to the filter element 50 described herein. For example, in some embodiments, the frame 404 comprises a straight piece of nitinol wire shape set into a circular or oblong hoop or hoop with one or more struts 408 extending between the hoop and a filter wire 412. The strut 408 may be free from a coupling with the filter element 406 such that a portion of the filter element 406 is free to extend into the aortic arch 30 and possibly into the descending aorta 28 while the strut 408 remains coupled to an actuation mechanism (which may be the filter wire 412) within the outer sheath 402, although this is not required. The frame 404 may form a shape of an opening 414 of the filter assembly 400. The opening 414 may be circular, elliptical, or any shape that can appropriately appose sidewalls of a vessel. The filter assembly 400 may have a generally distally-facing opening 414. In other embodiments, the opening 414 may be proximally facing. The orientation of the opening 414 may vary depending on where the access incision is located and/or the vessel in which the filter assembly 400 is to be positioned.

In some cases, the filter assembly 400 may include a self-expanding filter assembly (e.g., comprising a superelastic material with stress-induced martensite due to confinement in the outer sheath 402). The filter assembly 400 may comprise a shape-memory material configured to self-expand upon a temperature change (e.g., heating to body temperature). The filter assembly 400 may comprise a shape-memory or superelastic frame (e.g., comprising a distal end hoop comprising nitinol) and a microporous material (e.g., comprising a polymer including laser-drilled holes) coupled to the frame, for example similar to the filter assemblies described in U.S. Pat. No. 8,876,796.

The filter assembly 400 may be coupled (e.g., crimped, welded, soldered, etc.) to a distal end of a deployment wire or filter wire 412 via a strut or wire, such as, but not limited to the leg 408, although this is not required. When both or all of the filter wire 412 and the strut are provided, the filter wire 412 and the strut may be coupled proximal to the filter assembly 400 using a crimp mechanism. In other embodiments, the filter wire 412 and the strut 408 may be a single unitary structure. The filter wire 412 and/or strut 408 can comprise a rectangular ribbon, a round (e.g., circular, elliptical) filament, a portion of a hypotube, a braided structure (e.g., as described herein), combinations thereof, and the like. The filter wire 412 can be coupled to the handle (not explicitly shown) and/or a slider to provide differential longitudinal movement versus the outer sheath 402, which can sheathe and unsheathe the filter assembly 400 from the outer sheath 402.

The filter element 406 may comprise a “wind sock” shaped filter bag, the proximal end region 416 or base of which can separate from the outer sheath 402 and/or filter wire 412 and extend into and/or across the aortic arch 30. For example, the filter bag 406 may have a generally tubular shape with an enclosed proximal end region 416. The number and location of filter holes in the filter element 406 may be varied in order to optimize the pressure gradient across the filter element 406 to ensure good wall apposition and sealing. In one embodiment, a tether 410 may extend distally from a proximal end of the outer sheath 402 (for example, from a handle) to the filter assembly 400. A distal end of the tether 410 may be coupled to the base 416 of the filter element 406. The tether 410 may be formed of woven suture material or the like. The material of the tether 410 may be such that the tether 410 may be tensioned during retrieval of the filter assembly 400 to pull the base 416 of the filter element 406 back into the outer sheath 402 in order to collapse the filter assembly 400 and prevent bunching of filter element 406 during retrieval.

The delivery of the filter assembly 400 may be similar to the delivery of the filter assembly 66 described herein. In some methods of use, the filter assembly 400 may first be withdrawn into the outer sheath 402 to collapse the filter assembly 400 (not explicitly shown). The outer sheath 402 may be advanced to the target location with or without a guidewire, as desired. The filter system 420 (e.g., filter assembly 400, outer sheath 402, and other delivery elements) may be advanced into the subject through an incision made in the subject's right radial artery, or alternatively the right brachial artery. However, the system 420 may be advanced using an access location desired, including, but not limited to left radial access, left brachial access, femoral access, etc.

When placing the system 420, it may be desirable to avoid contact with the aortic valve to avoid damaging it. This may be accomplished by inserting a pigtail angiography catheter into the aortic arch through either a femoral access or through a left radial artery access, and injecting contrast so that the aortic valve may be located and imaged using fluoroscopy. However, it may be desired to not require the addition of femoral access for an angiography catheter given that femoral access is not required for all index procedure catheters, and femoral access is associated with increased access site vascular complications. It is contemplated that an angiography catheter (in some cases, a 4 Fr angiography catheter) may be advanced through an inner lumen of the outer sheath 402 and into the filter assembly 400 before or after deployment of the filter assembly 400. Contrast may then be injected through the angiography catheter. In another embodiment, contrast may be injected directly through the guidewire lumen of an inner member (if so provided) or through the inner lumen of the outer sheath 402 if the inner member is removed. In yet another embodiment, contrast may not be used and the filter assembly 400 may be placed with imaging using transesophageal echocardiography (TEE).

Once the system 420 is in a desired position, the guidewire (if used) may then be proximally retracted. The system 420 may be delivered to the ascending aorta 26 in a delivery configuration. The system's delivery configuration generally refers to the configuration where the filter assembly 400 is in a collapsed configuration within the outer sheath 402. The outer sheath 402 is retracted proximally to allow filter frame 404 to expand to an expanded configuration against the wall of the ascending aorta 26 upstream of the ostium of the innominate artery 12. The filter element 406 is secured either directly or indirectly to support frame 404 and is therefore reconfigured to the configuration shown in FIG. 5. Alternatively, or additionally, the filter assembly 400 may be distally advanced from the outer sheath 402 through distal actuation of the filter wire 412. Once expanded, the filter assembly 400 filters blood traveling through the ascending aorta 26, and therefore filters blood traveling into innominate artery 12, the right common carotid artery 18, the right vertebral artery 20, the left common carotid artery 14, the left subclavian artery 16, the left vertebral artery 24, and the descending aorta 28. The expanded filter assembly 400 is therefore in position to prevent foreign particles from traveling into all four cerebral arteries 14, 18, 20, 24, and into the cerebral vasculature.

The filter assembly 400 may be re-sheathed or positioned within the outer sheath 402 to facilitate repositioning of the filter assembly to optimize the placement of the filter assembly 400. To re-sheathe the filter assembly 400, it is contemplated that the outer sheath 402 may be distally advanced over the filter assembly 400, the filter assembly 400 proximally retracted into the outer sheath via the filter wire 408, or combinations thereof. The system 420 may then be repositioned and the filter assembly 400 redeployed. The filter assembly 400 may be re-sheathed, repositioned, and redeployed as many times as desired to optimize the placement of the filter assembly 400.

Once the filter assembly 400 has been deployed, the index procedure (e.g., LAOO, TMVR, ablation, etc.) may then be performed. Following the completion of the index procedure, the filter assembly 400 may be sheathed (e.g., collapsed within the outer sheath 402) and the system 420 removed, with any captured debris removed along with the filter assembly 400.

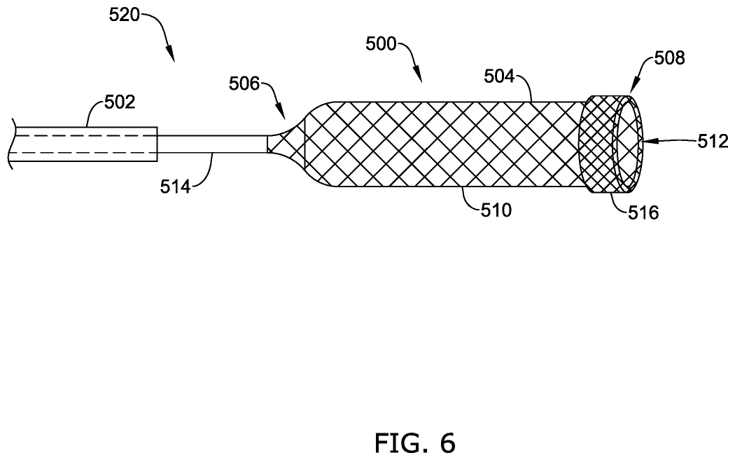

FIG. 6 is a partial perspective view of another illustrative filter assembly 500. The filter assembly 500 may be configured to be advanced to the target location within an outer sheath 502 that may be similar in form and function to the outer sheath 52 described with respect to FIG. 1. The filter assembly 500 may include an elongated tubular body 504. While the filter assembly 500 is described as generally tubular, it is contemplated that the filter assembly 500 may take any cross-sectional shape desired. The filter assembly 500 may have a first, or proximal end 506, a second, or distal end 508, and an intermediate region 510 disposed between the first end 506 and the second end 508. The filter assembly 500 may include a cavity 512 extending from an opening adjacent the distal end 508 to the proximal end 506. The proximal end 506 may be enclosed to trap particles within the cavity 512.

The filter assembly 500 may be expandable from a first radially collapsed configuration (not explicitly shown) to a second radially expanded configuration. In some cases, the filter assembly 500 may be deployed to a configuration between the collapsed configuration and a fully expanded configuration. In the expanded state, the tubular body 504 may be configured to filter fluid (e.g., blood) flowing through the tubular body 504 and to inhibit or prevent particles (e.g., embolic material) from flowing through the tubular body 504 by capturing the particles in the tubular body 504. The tubular body 504 may have a woven structure, fabricated from a number of filaments. In some embodiments, the tubular body 504 may be braided with one filament. In other embodiments, the tubular body 504 may be braided with several filaments. In another embodiment, the tubular body 504 may be knitted. In yet another embodiment, the tubular body 504 may be of a knotted type. In still another embodiment, the tubular body 504 may be laser cut.

In some embodiments, the tubular body 504 may be self-expanding (e.g., comprising a superelastic material with stress-induced martensite due to confinement in the outer sheath 502). The filter assembly 500 may comprise a shape-memory material configured to self-expand upon a temperature change (e.g., heating to body temperature). The filter assembly 500 may comprise a shape-memory or superelastic frame (e.g., comprising a distal end hoop comprising nitinol). The tubular body 504 may be woven, braided, knitted, knotted laser drilled, etc. such that the space between adjacent filaments (or other openings formed therein) are small enough to inhibit or prevent particles (e.g., embolic material) from flowing through the tubular body 504 by capturing the particles in the tubular body 504.

In some embodiments, the distal end 508 may have an outer diameter that is larger than an outer diameter of the intermediate region 510 and/or the proximal end 506. This may help provide good apposition with the wall when the filter assembly 500 is deployed. However, this is not required. In other embodiments, the distal end 508 and the intermediate region 510 may have a substantially uniform outer diameter. It is contemplated that the proximal end 506 may have a reduced diameter relative to the remaining portions of the tubular body 504 to create an enclosed end to trap the particles.

It is contemplated that the distal end 508 of the tubular body 504 may be sharp or jagged when wire filaments are used to form the tubular body 504. It is contemplated that the distal end 508 may include a polymer coating, such as but not limited to, polyurethane or silicone, to limit damage to the vasculature during deployment. Alternatively, or additionally, the distal end 508 may be folded back over itself to form a hem 516. The hem 516 may be mechanically secured using sutures, adhesives, etc., if so desired.

The filter assembly 500 may be coupled (e.g., crimped, welded, soldered, etc.) to a distal end of a deployment wire or filter wire 514. The filter wire 514 can comprise a rectangular ribbon, a round (e.g., circular, elliptical) filament, a portion of a hypotube, a braided structure (e.g., as described herein), combinations thereof, and the like. The filter wire 514 can be coupled to a handle (not explicitly shown) and/or a slider to provide differential longitudinal movement versus the outer sheath 502, which can sheathe and unsheathe the filter assembly 500 from the outer sheath 502.

The delivery of the filter assembly 500 may be similar to the delivery of the filter assembly 66 described herein. In some methods of use, the filter assembly 500 may first be withdrawn into the outer sheath 502 to collapse the filter assembly 500 (not explicitly shown). The outer sheath 502 may be advanced to the target location with or without a guidewire, as desired. The filter system 520 (filter assembly 500, outer sheath 502, and other delivery components) may be advanced into the subject through an incision made in the subject's right radial artery, or alternatively the right brachial artery. However, the system 520 may be advanced using an access location desired, including, but not limited to left radial access, left brachial access, femoral access, etc.

When placing the system 520, it may be desirable to avoid contact with the aortic valve to avoid damaging it. This may be accomplished by inserting a pigtail angiography catheter into the aortic arch through either a femoral access or through a left radial artery access, and injecting contrast so that the aortic valve may be located and imaged using fluoroscopy. However, it may be desired to not require the addition of femoral access for an angiography catheter given that femoral access is not required for all index procedure catheters, and femoral access is associated with increased access site vascular complications. It is contemplated that an angiography catheter (in some cases, a 4 Fr angiography catheter) may be advanced through an inner lumen of the outer sheath 502 and into the filter assembly 500 before or after deployment of the filter assembly 500. Contrast may then be injected through the angiography catheter. In another embodiment, contrast may be injected directly through the guidewire lumen of an inner member (if so provided) or through the inner lumen of the outer sheath 502 if the inner member is removed. In yet another embodiment, contrast may not be used and the filter assembly 500 may be placed with imaging using transesophageal echocardiography (TEE).

Once the system 520 is in a desired position, the guidewire (if used) may then be proximally retracted. The system 520 may be delivered to the ascending aorta (see, for example, FIG. 1) in a delivery configuration. The system's delivery configuration generally refers to the configuration where the filter assembly 500 is in a collapsed configuration within the outer sheath 502. The outer sheath 502 is retracted proximally to allow the tubular body 504 to expand to an expanded configuration against the wall of the ascending aorta upstream of the ostium of the innominate artery. Alternatively, or additionally, the filter assembly 500 may be distally advanced from the outer sheath 502 through distal actuation of the filter wire 514. Once expanded, the filter assembly 500 filters blood traveling through the ascending aorta, and therefore filters blood traveling into innominate artery, the right common carotid artery, the right vertebral artery, the left common carotid artery, the left subclavian artery, the left vertebral artery, and the descending aorta. The expanded filter assembly 500 is therefore in position to prevent foreign particles from traveling into all four cerebral arteries, and into the cerebral vasculature.

The filter assembly 500 may be re-sheathed or positioned within the outer sheath 502 to facilitate repositioning of the filter assembly 500 to optimize the placement of the filter assembly 500. To re-sheathe the filter assembly 500, it is contemplated that the outer sheath 502 may be distally advanced over the filter assembly 500, the filter assembly 500 proximally retracted into the outer sheath 502 via the filter wire 514, or combinations thereof. The system 520 may then be repositioned and the filter assembly 500 redeployed. The filter assembly 500 may be re-sheathed, repositioned, and redeployed as many times as desired to optimize the placement of the filter assembly 500.

Once the filter assembly 500 has been deployed, the index procedure (e.g., LAOO, TMVR, ablation, etc.) may then be performed. Following the completion of the index procedure, the filter assembly 500 may be sheathed (e.g., collapsed within the outer sheath 502) and the system 520 removed, with any captured debris removed along with the filter assembly 500.

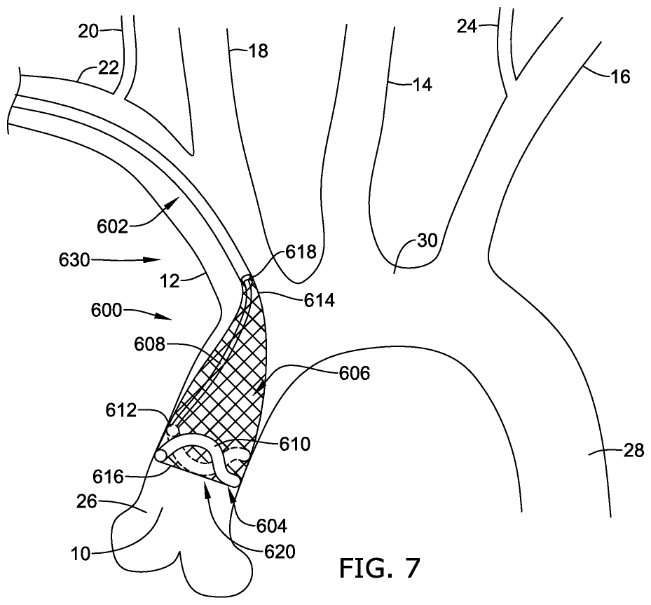

FIG. 7 is a schematic view of another illustrative filter assembly 600 positioned within the ascending aorta 26. The filter assembly 600 may be configured to be advanced to the target location within an outer sheath 602 that may be similar in form and function to the outer sheath 52 described with respect to FIG. 1. The filter assembly 600 may include a variable diameter support element or frame 604 and a filter element 606. The frame 604 may be an expandable structure configured to generally provide expansion support to the filter element 606 in the expanded state. In the expanded state, the filter element 606 may be configured to filter fluid (e.g., blood) flowing through the filter element 606 and to inhibit or prevent particles (e.g., embolic material) from flowing through the filter element 606 by capturing the particles in the filter element 606. The filter element 606 may be similar in form and function to the filter element 50 described herein. While not explicitly shown, the filter assembly 600 may include any of the additional support elements described herein, such as, but not limited to, one or more longitudinally extending tines, an elongated hoop, a hemmed terminal end, etc. It is further contemplated that the filter assembly 600 may include a tether coupled to the base of the filter element 606 such that the base may be drawn into the outer sheath 602 upon actuation of the tether.

The frame 604 may be formed from an inflatable fabric or polymer tube. The frame 604 may be expandable from a first collapsed configuration to a second expanded configuration through the injection of an inflation media into a sealable and enclosed inflation cavity 610 in the frame 604 such that the diameter of the frame 604 is variable. For example, the diameter can be increased by injection more inflation fluid and reduced by removing inflation fluid (or injecting less). The inflation cavity 610 may receive an inflation fluid from an inflation fluid source through an inflation port or valve 612 to expand the frame 604 from a generally collapsed delivery configuration (not explicitly shown) to an expanded or deployed configuration (as shown in FIG. 7). The inflation fluid may be saline, a biocompatible liquid polymer, such as ENTERYX®, air, or other suitable inflation fluid.

While the frame 604 is illustrated as having an undulating configuration, other configuration as also contemplated. For example, the frame 604 may take the form of a ring or hoop. Alternatively or additionally, the frame 604 may include longitudinally extending portions. In some instances, the frame 604 may be a helical frame, winding about the circumference of filter assembly 600 from a first end 614 to a second end 616. It is contemplated that a plurality of inflation chambers may be fluidly connected to allow a single inflation valve 612 to provide an inflation fluid to each of the chambers. However, this is not required. A plurality of inflation valves may be provided to supply each of the inflation chambers, individually or in groups, with an inflation fluid. These are just examples.

The inflation valve 612 may be in fluid communication with the inflation cavity 610 to provide a regulated passage for an inflation fluid to travel into the inflation cavity 610 of the frame 604. The inflation valve 612 may be any of a number of widely applied valves, applicable in surgeries and medical implants, and may be made from a biocompatible material. In some embodiments, the inflation valve 612 may be a unidirectional, or one-way, valve that provides a regulated passage for an amount of a suitable fluid into the inflation cavity 610 of the inflatable frame 604. For example, the inflation valve 612 may provide such a passage upon an application of pressure from a catheter lumen or an inflation device that is introduced into the frame 604 for the stent's inflation. Once the application of pressure is removed, a diaphragm or other sealing mechanism may seal the inflation cavity 610 to maintain the frame 604 in the inflated state. However, this is not required. In some embodiments, inflation fluid may be continually supplied to the inflation cavity 610 to maintain the frame 604 in an expanded configuration. For example, inflation fluid may be continuously delivered to the inflation cavity 610 via a constant pressure source. For example, inflation fluid may be continuously delivered to maintain a measured pressure at a constant (or approximately constant value). Such a system may help maintain apposition of the frame 604 with a vessel wall if there is compliance and/or stretching in the frame 604 over the course of the procedure.

The inflation cavity 610 and/or inflation valve 612 may be in fluid communication with an inflation fluid source via an inflation lumen 608. The inflation lumen 608 may be a dedicated lumen extending proximally from the inflation cavity 610 to the proximal end region of the system 630 for receiving the inflation fluid. It is contemplated that the inflation fluid may be injected from a handle, through the inflation lumen 608 and to the inflation cavity 610 to expand the frame 604 and seal it against the vessel wall. In some embodiments, suction may be applied to the inflation lumen 608 to remove the inflation fluid from the inflation cavity 610 and collapse the frame 604. This may be performed to reposition and/or remove the filter assembly 600.

In some embodiments, the filter assembly 600 may be coupled to an elongate shaft and/or filter wire 618 adjacent to the first end 614 of the filter assembly 600. It is contemplated that the inflation lumen 608 may extend within the elongate shaft 618 or alongside the elongate shaft 618, as desired. The elongate shaft 618 can be coupled to a handle (not explicitly shown) and/or a slider to provide differential longitudinal movement versus the outer sheath 602, which can sheathe and unsheathe the filter assembly 600 from the outer sheath 602.

The frame 604 may form a shape of an opening 620 of the filter assembly 600. The opening 620 may be circular, elliptical, or any shape that can appropriately appose sidewalls of a vessel. The filter assembly 600 may have a generally distally-facing opening 620. In other embodiments, the opening 620 may be proximally facing. The orientation of the opening 620 may vary depending on where the access incision is located and/or the vessel in which the filter assembly 600 is to be positioned.

The delivery of the filter assembly 600 may be similar to the delivery of the filter assembly 66 described herein. In some methods of use, the filter assembly 600 may first be withdrawn into the outer sheath 602 for delivery. The outer sheath 602 may be advanced to the target location with or without a guidewire, as desired. The filter system 630 (filter assembly 600, outer sheath 602, and other delivery components) may be advanced into the subject through an incision made in the subject's right radial artery, or alternatively the right brachial artery. However, the system 630 may be advanced using an access location desired, including, but not limited to left radial access, left brachial access, femoral access, etc.

When placing the system 630, it may be desirable to avoid contact with the aortic valve to avoid damaging it. This may be accomplished by inserting a pigtail angiography catheter into the aortic arch through either a femoral access or through a left radial artery access, and injecting contrast so that the aortic valve may be located and imaged using fluoroscopy. However, it may be desired to not require the addition of femoral access for an angiography catheter given that femoral access is not required for all index procedure catheters, and femoral access is associated with increased access site vascular complications. It is contemplated that an angiography catheter (in some cases, a 4 Fr angiography catheter) may be advanced through an inner lumen of the outer sheath 602 and into the filter assembly 600 before or after deployment of the filter assembly 600. Contrast may then be injected through the angiography catheter. In another embodiment, contrast may be injected directly through the guidewire lumen of an inner member (if so provided) or through the inner lumen of the outer sheath 602 if the inner member is removed. In yet another embodiment, contrast may not be used and the filter assembly 600 may be placed with imaging using transesophageal echocardiography (TEE).

Once the system 630 is in a desired position, the guidewire (if used) may then be proximally retracted. The system 630 may be delivered to the ascending aorta 26 in a delivery configuration. The system's delivery configuration generally refers to the configuration where the filter assembly 600 is in a collapsed configuration within the outer sheath 602. The outer sheath 602 is retracted proximally and inflation fluid is injected into the inflation cavity 610 to allow filter frame 604 to expand to an expanded configuration against the wall of the ascending aorta 26 upstream of the ostium of the innominate artery 12. The filter element 606 is secured either directly or indirectly to support frame 604 and is therefore reconfigured to the configuration shown in FIG. 7. Alternatively, or additionally, the filter assembly 600 may be distally advanced from the outer sheath 602 through distal actuation of the elongate shaft 618 and inflation fluid delivered to the inflation cavity 610. Once expanded, the filter assembly 600 filters blood traveling through the ascending aorta 26, and therefore filters blood traveling into innominate artery 12, the right common carotid artery 18, the right vertebral artery 20, the left common carotid artery 14, the left subclavian artery 16, the left vertebral artery 24, and the descending aorta 28. The expanded filter assembly 600 is therefore in position to prevent foreign particles from traveling into all four cerebral arteries 14, 18, 20, 24, and into the cerebral vasculature.

The filter assembly 600 may be re-sheathed or positioned within the outer sheath 602 to facilitate repositioning of the filter assembly to optimize the placement of the filter assembly 600. To re-sheathe the filter assembly 600, it is contemplated that the inflation fluid is removed from the inflation cavity 610 and the outer sheath 602 distally advanced over the filter assembly 600, the filter assembly 600 proximally retracted into the outer sheath via the elongate shaft 618, or combinations thereof. The system 630 may then be repositioned and the filter assembly 600 redeployed. The filter assembly 600 may be re-sheathed, repositioned, and redeployed as many times as desired to optimize the placement of the filter assembly 600.

Once the filter assembly 600 has been deployed, the index procedure (e.g., LAOO, TMVR, ablation, etc.) may then be performed. Following the completion of the index procedure, the filter assembly 600 may be sheathed (e.g., collapsed within the outer sheath 602) and the system 630 removed, with any captured debris removed along with the filter assembly 600.

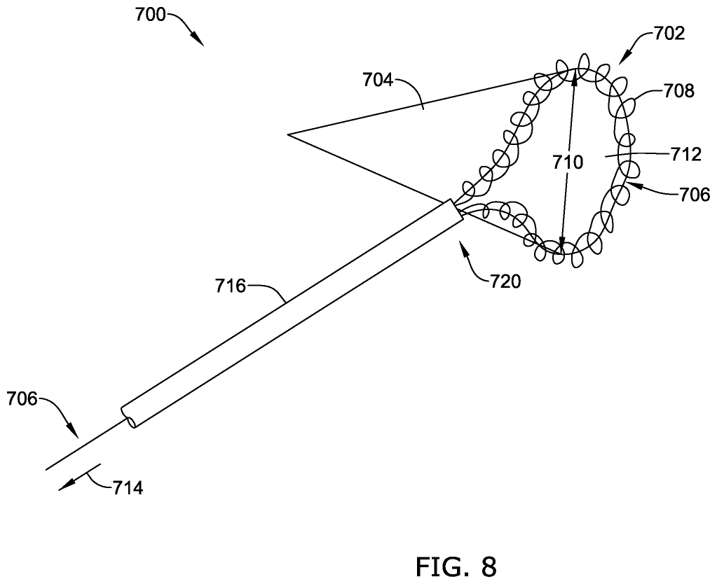

FIG. 8 is a partial schematic view of another illustrative filter assembly 700. The filter assembly 700 may be configured to be advanced to the target location within an outer sheath 716 that may be similar in form and function to the outer sheath 52 described with respect to FIG. 1. The filter assembly 700 may include a support element or frame 702 and a filter element 704. The frame 702 may be an expandable structure configured to generally provide expansion support to the filter element 704 in the expanded state. In the expanded state, the filter element 704 may be configured to filter fluid (e.g., blood) flowing through the filter element 704 and to inhibit or prevent particles (e.g., embolic material) from flowing through the filter element 704 by capturing the particles in the filter element 704. The filter element 704 may be similar in form and function to the filter element 50 described herein. While not explicitly shown, the filter assembly 700 may include any of the additional support elements described herein, such as, but not limited to, one or more longitudinally extending tines, an elongated hoop, a hemmed terminal end, etc. It is further contemplated that the filter assembly 700 may include a tether coupled to the base of the filter element 704 such that the base may be drawn into the outer sheath 716 upon actuation of the tether.

In some embodiments, the frame 702 may be self-expanding (e.g., comprising a superelastic material with stress-induced martensite due to confinement in the outer sheath 716). The filter assembly 700 may comprise a shape-memory material configured to self-expand upon a temperature change (e.g., heating to body temperature). The filter assembly 700 may comprise a shape-memory or superelastic frame (e.g., comprising a distal end hoop comprising nitinol). The frame 702 may be formed from an open spaced coil including a plurality of windings 708 which can be expanded or contracted to change a diameter 710 of the opening 712 of the frame 702 such that the diameter of the frame 702 is variable. Alternatively, the frame 702 may be formed from a laser cut tube having a plurality of openings. The frame 702 may have a generally circular shape. A flexible tension element 706 may be extend through or be threaded through a central lumen of the frame 702. For example, when the frame 702 includes a plurality of windings, the flexible tension element 706 may be extend through or be threaded within a center of the plurality of windings 708. A distal end of the flexible tension element 706 may be fixedly coupled to the frame 702 such that a proximal or pulling force 714 applied to a proximal end of the tension element 706 (the proximal end extending proximally from the frame 702 and configured to remain outside the body) exerts a radially inward force of the frame and draws the windings 708 closer together to reduce the diameter 710 of the frame 702. The diameter 710 of the frame 702 may be increased by relaxing tension on the flexible tension element 706 to allow the frame 702 to expand. It is contemplated that the pitch of the coil or laser cut element can vary such that different regions will elongate or foreshorten as the internal tension element 706 is adjusted by the operator resulting in a more optimal seal against the vessel wall. In some embodiments, the internal tension element 706 may be coupled to a device configured to exert a constant or nearly constant pressure on the internal tension element 706. For example, the internal tension element 706 may be coupled to a spring in a handle, or other mechanism, configured to achieve a nearly constant pressure on the internal tension element 706.

In some embodiments, the filter assembly 700 may be coupled to the tension element 706. The tension element 706 can be coupled to a handle (not explicitly shown) and/or a slider to provide differential longitudinal movement versus the outer sheath 716, which can sheathe and unsheathe the filter assembly 700 from the outer sheath 716. In other embodiments, a filter wire (not explicitly shown) may be coupled to the filter assembly 700 to actuate the filter assembly 700 in a proximal and/or distal direction while the tension element 706 is actuated to control a diameter 710 of the filter frame 702.

The frame 702 may form a shape of an opening 712 of the filter assembly 700. The opening 712 may be circular, elliptical, or any shape that can appropriately appose sidewalls of a vessel. The filter assembly 700 may have a generally distally-facing opening 712. In other embodiments, the opening 712 may be proximally facing. The orientation of the opening 712 may vary depending on where the access incision is located and/or the vessel in which the filter assembly 700 is to be positioned.

The delivery of the filter assembly 700 may be similar to the delivery of the filter assembly 66 described herein. In some methods of use, the filter assembly 700 may first be withdrawn into the outer sheath 716 for delivery. The outer sheath 716 may be advanced to the target location with or without a guidewire, as desired. The filter system 720 (filter assembly 700, outer sheath 716, and other delivery components) may be advanced into the subject through an incision made in the subject's right radial artery, or alternatively the right brachial artery. However, the system 720 may be advanced using an access location desired, including, but not limited to left radial access, left brachial access, femoral access, etc.

When placing the system 720, it may be desirable to avoid contact with the aortic valve to avoid damaging it. This may be accomplished by inserting a pigtail angiography catheter into the aortic arch through either a femoral access or through a left radial artery access, and injecting contrast so that the aortic valve may be located and imaged using fluoroscopy. However, it may be desired to not require the addition of femoral access for an angiography catheter given that femoral access is not required for all index procedure catheters, and femoral access is associated with increased access site vascular complications. It is contemplated that an angiography catheter (in some cases, a 4 Fr angiography catheter) may be advanced through an inner lumen of the outer sheath 716 and into the filter assembly 700 before or after deployment of the filter assembly 700. Contrast may then be injected through the angiography catheter. In another embodiment, contrast may be injected directly through the guidewire lumen of an inner member (if so provided) or through the inner lumen of the outer sheath 716 if the inner member is removed. In yet another embodiment, contrast may not be used and the filter assembly 700 may be placed with imaging using transesophageal echocardiography (TEE).

Once the system 720 is in a desired position, the guidewire (if used) may then be proximally retracted. The system 720 may be delivered to the ascending aorta (see, for example, FIG. 1) in a delivery configuration. The system's delivery configuration generally refers to the configuration where the filter assembly 700 is in a collapsed configuration within the outer sheath 716. The outer sheath 716 is retracted proximally and the filter frame 702 to expands to an expanded configuration against the wall of the ascending aorta upstream of the ostium of the innominate artery. A proximal force may be exerted on the tension element 706 to reduce the diameter 710 of the opening or a distal pushing force exerted (or the proximal force simply removed) on the tension element 706 to increase the diameter 710 of the opening 712. The filter element 704 is secured either directly or indirectly to support frame 702 and is therefore reconfigured to the configuration shown in FIG. 8. Alternatively, or additionally, the filter assembly 700 may be distally advanced from the outer sheath 716 through distal actuation of the filter wire. Once expanded, the filter assembly 700 filters blood traveling through the ascending aorta, and therefore filters blood traveling into innominate artery, the right common carotid artery, the right vertebral artery, the left common carotid artery, the left subclavian artery, the left vertebral artery, and the descending aorta. The expanded filter assembly 700 is therefore in position to prevent foreign particles from traveling into all four cerebral arteries, and into the cerebral vasculature.

The filter assembly 700 may be re-sheathed or positioned within the outer sheath 716 to facilitate repositioning of the filter assembly to optimize the placement of the filter assembly 700. To re-sheathe the filter assembly 700, it is contemplated that the outer sheath 716 may be distally advanced over the filter assembly 700, the filter assembly 700 proximally retracted into the outer sheath 716 via the filter wire, or combinations thereof. The system 720 may then be repositioned and the filter assembly 700 redeployed. The filter assembly 700 may be re-sheathed, repositioned, and redeployed as many times as desired to optimize the placement of the filter assembly 700.

Once the filter assembly 700 has been deployed, the index procedure (e.g., LAOO, TMVR, ablation, etc.) may then be performed. Following the completion of the index procedure, the filter assembly 700 may be sheathed (e.g., collapsed within the outer sheath 716) and the system 720 removed, with any captured debris removed along with the filter assembly 700.

While the methods and devices described herein may be susceptible to various modifications and alternative forms, specific examples thereof have been shown in the drawings and are described in detail herein. It should be understood, however, that the inventive subject matter is not to be limited to the particular forms or methods disclosed, but, to the contrary, covers all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. In any methods disclosed herein, the acts or operations can be performed in any suitable se and are not necessarily limited to any particular disclosed sequence and not be performed in the order recited. Various operations can be described as multiple discrete operations in turn, in a manner that can be helpful in understanding certain embodiments; however, the order of description should not be construed to imply that these operations are order dependent. Additionally, the structures described herein can be embodied as integrated components or as separate components. For purposes of comparing various embodiments, certain aspects and advantages of these embodiments are described. Not necessarily all such aspects or advantages are achieved by any particular embodiment. Thus, for example, embodiments can be carried out in a manner that achieves or optimizes one advantage or group of advantages without necessarily achieving other advantages or groups of advantages. The methods disclosed herein may include certain actions taken by a practitioner; however, the methods can also include any third-party instruction of those actions, either expressly or by implication. For example, actions such as “deploying a self-expanding filter” include “instructing deployment of a self-expanding filter.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 7 mm” includes “7 mm.” Phrases preceded by a term such as “substantially” include the recited phrase and should be interpreted based on the circumstances (e.g., as much as reasonably possible under the circumstances). For example, “substantially straight” includes “straight.”

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A method of inhibiting embolic material from entering cerebral vasculature, the method comprising:
positioning a guidewire through a right subclavian artery and into an aortic arch;
tracking a distal portion of a protection device over the guidewire, the distal portion of the protection device comprising:
a proximal sheath;
a proximal self-expanding filter assembly radially within the proximal sheath;
a distal sheath; and
a distal self-expanding filter assembly radially within the distal sheath;
at least one of proximally retracting the proximal sheath and distally advancing the proximal self-expanding filter assembly to deploy the proximal self-expanding filter assembly from the proximal sheath in an innominate artery;
steering the distal sheath into the aortic arch;
at least one of proximally retracting the distal sheath and distally advancing the distal self-expanding filter assembly to deploy the distal self-expanding filter assembly from the distal sheath in the aortic arch; and
after deploying the proximal self-expanding filter assembly and distal self-expanding filter assembly, withdrawing the proximal sheath and the distal sheath.

2. The method of claim 1, wherein an opening of the distal self-expanding filter assembly is positioned in the aortic arch upstream of an ostium of a left common carotid artery.

3. The method of claim 2, wherein the opening is a distally facing opening.

4. The method of claim 1, wherein the distal self-expanding filter assembly comprises a frame, a filter element, and a support element.

5. The method of claim 4, wherein the support element is one or more longitudinally extending tines.

6. The method of claim 4, wherein the support element is an elongated hoop positioned between the frame and a base of the filter element.

7. A method of inhibiting embolic material from entering cerebral vasculature, the method comprising:
positioning a guidewire through a right subclavian artery and into an ascending aorta;
tracking a distal portion of a protection device over the guidewire, the distal portion of the protection device comprising:
an outer sheath; and
a self-expanding filter assembly radially within the outer sheath; and
at least one of proximally retracting the outer sheath and distally advancing the self-expanding filter assembly to deploy the self-expanding filter assembly from the outer sheath in the ascending aorta.

8. The method of claim 7, wherein the self-expanding filter assembly comprises a frame, a filter element, and a support element.

9. The method of claim 8, wherein the support element is one or more longitudinally extending tines.

10. The method of claim 8, wherein the support element is an elongated hoop positioned between the frame and a base of the filter element.

11. The method of claim 7, wherein the self-expanding filter assembly further comprises a tether coupled to a base of a filter element.

12. The method of claim 11, wherein proximal actuation of the tether draws the base of the filter element into the outer sheath.

13. The method of claim 7, wherein the self-expanding filter assembly comprises an elongated tubular body.

14. The method claim 13, wherein the elongated tubular body comprises one or more woven, braided, or knitted filaments.

15. The method of claim 14, wherein a distal end of the elongated tubular body comprises a hem.

16. A method of inhibiting embolic material from entering cerebral vasculature, the method comprising:
positioning a guidewire through a right subclavian artery and into an ascending aorta;
tracking a distal portion of a protection device over the guidewire, the distal portion of the protection device comprising:
an outer sheath; and
an inflatable filter assembly radially within the outer sheath;
at least one of proximally retracting the outer sheath and distally advancing the inflatable filter assembly to deploy the inflatable filter assembly from the outer sheath in the ascending aorta; and
delivering an inflation fluid to the inflatable filter assembly to expand a frame of the inflatable filter assembly.

17. The method of claim 16, wherein the frame of the inflatable filter assembly comprises an inflation cavity and a valve.

18. The method of claim 17, wherein the inflation cavity is fluidly coupled to an inflation lumen.

19. The method of claim 16, wherein the inflatable filter assembly further comprises a filter element coupled to the frame.

20. The method of claim 16, further comprising removing the inflation fluid.
